(19) United States
(12) Patent Application Publication
(10) Pub. No.: US 2024/0326043 A1
(43) Pub. Date: Oct. 3, 2024

(54) SYSTEMS, DEVICES, AND METHODS FOR FLUID TRANSFER WITHIN AN AUTOMATED CELL PROCESSING SYSTEM

(71) Applicant: Cellares Corporation, South San Francisco, CA (US)

(72) Inventors: Fabian GERLINGHAUS, South San Francisco, CA (US); Andrew Philip Gust PETERSON, San Francisco, CA (US); Joshua SHEN, Castro Valley, CA (US); Bharat S. THAKKAR, Campbell, CA (US); Joon Mo YANG, Redwood City, CA (US)

(21) Appl. No.: 18/620,826

(22) Filed: Mar. 28, 2024

Related U.S. Application Data
(60) Provisional application No. 63/456,388, filed on Mar. 31, 2023.

(51) Int. Cl. B01L 3/00; B67D 3/00; B67D 7/02

(52) U.S. Cl. CPC B01L 3/502715; B67D 3/0061; B67D 7/0288; B01L 2300/049

(57) ABSTRACT

The present disclosure relates to systems, devices, and methods for automated fluid transfer. In an embodiment, the present disclosure relates to a system for automated fluid transfer, comprising a fluid pump, a fluid device, comprising a container for a volume of fluid, and a universal collar couplable to the container, the collar comprising a plurality of conduits, a sterile liquid transfer port in fluid communication with the plurality of conduits, a fluid pump module comprising compressible fluidic tubing coupled between an inlet port and an outlet port, each of the inlet port and the outlet port being in fluid communication with the plurality of conduits, and one or more viewing windows, and one or more sensors configured to detect, via the one or more viewing windows, the presence of liquid within a segment of the plurality of conduits.

### CROSS REFERENCE TO RELATED APPLICATIONS

This application claims priority to U.S. Provisional Patent Application No. 63/456,388, filed Mar. 31, 2023, the content of which is hereby incorporated in its entirety by this reference.

### TECHNICAL FIELD

The present disclosure relates to fluid transfer within an automated cell processing system.

### BACKGROUND

Cell therapy, where cells from an individual patient are collected, processed ex vivo, and then returned to the same patient, has been revolutionary for producing durable and effective clinical responses in patients. However appealing, cell therapy manufacturing is a complex, often labor-intensive process that is difficult to “scale-up” and is prone to human error and contamination. While recent efforts have been made toward automating manufacturing of cell therapies, such as automating movements of cells between manufacturing steps, traditional cell therapy manufacturing includes remain numerous inefficiencies. For example, the transfer of fluids between cell therapy steps and reagent storage devices continues to be a human touch point and an entryway for error and contamination. Given the importance of sterility in the transferring of fluids to collect samples, replenish culture media, and the like, additional systems, devices, and methods for fluid transfer within a cell processing system are desirable. In particular, automated systems, devices, and methods, and methods that can be performed in a sterile manner are desirable.

### SUMMARY

The present disclosure relates generally to systems, devices, and methods for automated fluid transfer within an automated cell processing system. In general, the fluid devices disclosed herein may comprise a container for a volume of fluid and a universal collar couplable to the container. In some variations, the collar comprises a plurality of conduits, a sterile liquid transfer port in fluid communication with the plurality of conduits, and a fluid pump module comprising compressible fluidic tubing coupled between an inlet port and an outlet port. Each of the inlet port and the outlet port may be in fluid communication with the plurality of conduits and the compressible fluidic tubing may be compressed by a fluid pump to control movement of fluids out of the container. In some variations, the collar may further comprise one or more sterilization process ports in fluid communication with the plurality of conduits. Sterilant, which may be one or more of vaporized hydrogen peroxide, ionized hydrogen peroxide, chlorine dioxide, and ethylene oxide, may be provided via the one or more sterilization process ports. In some variations, the collar may further comprise one or more air process ports in fluid communication with the plurality of conduits. The one or more air process ports may be compressed air process ports or air vents. In some variations, the collar may further comprise viewing windows that permit optical evaluation of fluid within a fluid conduit of the collar. In some variations, the sterile liquid transfer port may further comprise a mechanical seal. In some variations, the collar may further comprise one or more sterilization process ports in fluid communication with the plurality of conduits. In some variations, the mechanical seal of the sterile liquid transfer port and a sterilant provided via the one or more sterilization process ports ensure sterility of the collar. In some variations, the container comprises an opening and the collar may further comprise a fluid transport feature couplable to the opening, where the fluid transport feature may also be in fluid communication with the plurality of conduits. A venting tube may be configured to extend through the opening of the container and to be disposed within the container. The venting tube may further comprise a liquid vent reservoir configured to capture, upon inversion of the fluid device, fluid trapped within the venting tube. An air process tube may be configured to extend through the opening of the container and to be disposed within the container. In some variations, the sterile liquid transfer port may further comprise a mechanical seal and the collar may further comprise one or more sterilization ports in fluid communication with the plurality of conduits, the mechanical seal providing a first mechanism to achieve sterilization and the sterilant provided via the one or more sterilization ports providing a second mechanism to achieve sterilization.

Another fluid device for automated fluid transfer may include a container for a volume of fluid and a universal collar couplable to the container. The collar may include a plurality of conduits, a sterile liquid transfer port in fluid communication with the plurality of conduits, a fluid pump module, an air process port, and a ball valve coupled to the air process port. The fluid pump module may include compressible fluidic tubing coupled between an inlet port and an outlet port, where each of the inlet port and the outlet port may be in fluid communication with the plurality of conduits and the compressible fluidic tubing may be configured to be compressed by the fluid pump to control movement of fluids out of the container. The fluid device may be configured to be positioned in an upright orientation and an inverted orientation, and the ball valve may be configured to prevent the fluid of the container from flowing within the air process port when the fluid device is in the inverted orientation.

Methods for automated fluid transfer are also disclosed herein. In some variations, a method for automated fluid transfer comprises inverting, by a robot, a fluid device comprising a container and a universal collar comprising a plurality of conduits and a sterile liquid transfer port in fluid communication with the plurality of conduits; connecting, by the robot, the sterile liquid transfer port of the inverted fluid device to a corresponding sterile liquid transfer port of a cartridge; and pumping, via the plurality of conduits and the sterile liquid transfer port, fluid from the fluid device to the cartridge. In some variations, the method may further comprise sterilizing, after the connecting and before the pumping, the sterile liquid transfer port via one or more sterilization process ports of the collar in fluid communication with the plurality of conduits. In some variations, the method may further comprise actuating, by the robot and after the sterilizing, a valve of each of the sterile liquid transfer port and the corresponding sterile liquid transfer port to permit the pumping therethrough.

In other variations, a method for automated fluid transfer comprises inverting, by a robot, a fluid device comprising a container and a universal collar comprising a plurality of conduits, a sterile liquid transfer port, and an air process port, each of the sterile liquid transfer port and the air process port being in fluid communication with the plurality of conduits; connecting, by the robot, the sterile liquid transfer port of the inverted fluid device to a corresponding sterile liquid transfer port of a cartridge; pumping, via the plurality of conduits and the sterile liquid transfer port, at least a portion of a fluid from the inverted fluid device to the cartridge; and purging the plurality of conduits after the pumping using compressed air via the air process port.

In other variations, a method for automated fluid transfer comprises inverting, by a robot, a fluid device comprising a container and a universal collar comprising a robot engagement feature, a plurality of conduits, a sterile liquid transfer port, and a plurality of sterilization process ports, each of the sterile liquid transfer port and the plurality of sterilization process ports being in fluid communication with the plurality of conduits; connecting, by the robot, the sterile liquid transfer port of the inverted fluid device to a corresponding sterile liquid transfer port of a cartridge; flowing sterilant through the sterile liquid transfer port via the one or more sterilization process ports; and pumping, via the plurality of conduits and the sterile liquid transfer port, at least a portion of a fluid from the inverted fluid device to the cartridge.

In other variations, a method for automated fluid transfer comprises filling, when the fluid device is in an upright position, a fluid device comprising a container and a universal collar comprising a robot engagement feature, a plurality of conduits, and a sterile liquid transfer port, inverting, by a robot via the robot engagement feature, the fluid device; connecting, by the robot, the sterile liquid transfer port of the inverted fluid device to a corresponding sterile liquid transfer port of a cartridge; and pumping, via the plurality of conduits and the sterile liquid transfer device, at least a portion of a fluid from the fluid device to the cartridge. The pumping may further comprise receiving data from sensors arranged proximate to viewing windows of the collar, the sensors configured to detect the presence of liquid within a segment of the plurality of conduits, detecting, based on the received data, an air to liquid fluid transition, operate fluid pump based on the detected presence of the air to liquid fluid transition, detecting, based on the received data, a liquid to air fluid transition, and stopping operation of the fluid pump when the liquid to air fluid transition is detected. In some variations, the pumping may further comprise receiving data regarding a prescribed volume of fluid to transfer to the cartridge, where the data is received from sensors arranged proximate to viewing windows of the collar. In some variations, the sensors may be configured to detect the presence of liquid within a segment of the plurality of conduits. An air to liquid fluid transition may be detected based upon the received data, and a fluid pump may be operated to deliver the prescribed volume of fluid. Once the prescribed volume of fluid has been transferred, the operation of the fluid pump may be stopped.

In other variations, a method for automated fluid transfer comprises connecting, by a robot, a sterile liquid transfer port of a fluid device to a corresponding sterile liquid transfer port of a cartridge, pumping, via a plurality of conduits of the fluid device and the sterile liquid transfer port, at least a portion of a fluid from the fluid device to the cartridge and purging the plurality of conduits after the pumping using compressed air via an air process port of the fluid device.

Systems for automated fluid transfer are also disclosed herein. In some variations, a system for automated fluid transfer comprises a fluid pump, a fluid device comprising a container for a volume of fluid and a universal collar couplable to the container, and one or more sensors. The universal collar may comprise a plurality of conduits, a sterile liquid transfer port in fluid communication with the plurality of conduits, and one or more windows. The one or more sensors may be configured to detect the presence of liquid within a segment of the plurality of conduits via the one or more viewing windows. The collar may further comprise a fluid pump module comprising compressible fluidic tubing coupled between an inlet port and an outlet port, each of the inlet port and the outlet port being in fluid communication with the plurality of conduits, the compressible fluidic tubing being configured to be compressed by the fluid pump to control movement of fluids out of the container. In some variations, the system may further comprise a processor configured to receive data from the one or more sensors, detect, based on the received data, a fluid transition from air to liquid, start the fluid pump, detect a fluid transition from liquid to air, and stop the fluid pump when the fluid transition from liquid to air is detected.

Additional variations, features, and advantages of the invention will be apparent from the following detailed description and through practice of the invention.

### BRIEF DESCRIPTION OF THE DRAWINGS

FIG. TA is a block diagram of an illustrative variation of a cell processing system.

FIG. 1B is a block diagram of an illustrative variation of a cartridge.

FIG. 2A is a block diagram of an illustrative variation of a cell processing system. FIG. 2B is a perspective view of an illustrative variation of a workcell of a cell processing system. FIG. 2C is a perspective view of an illustrative variation of a workcell and cartridge of a cell processing system. FIG. 2D is a block diagram of an illustrative variation of a cell processing system.

FIG. 3A is a schematic diagram of an illustrative variation of a fluid device showing a container and a collar.

FIG. 3B is a schematic diagram of an illustrative system for automated fluid transfer.

FIG. 4A is a rendering of a perspective view of an illustrative fluid device for automated fluid transfer. FIG. 4B is a rendering of a perspective view of an illustrative collar of a fluid device for automated fluid transfer. FIG. 4C is a rendering of a top view of an illustrative collar of a fluid device for automated fluid transfer. FIG. 4D is an image of a bottom view of an illustrative collar of a fluid device for automated fluid transfer. FIG. 4E is an image of a view of an illustrative container and a fluid transport feature of a fluid device for automated fluid transfer. FIG. 4F is a rendering of a perspective view of an illustrative container of a fluid device for automated fluid transfer. FIG. 4G is a rendering of an illustrative fluid device for automated fluid transfer and a robot grasping feature of a robot of a workcell. FIG. 4H and FIG. 4I are renderings of an illustrative robot grasping feature of a robot of a workcell.

FIG. 5A is a rendering of a first perspective view of an illustrative fluid device for automated fluid transfer. FIG. 5B is a rendering of a second perspective view of an illustrative fluid device for automated fluid transfer. FIG. 5C is a rendering of a top view of an aspect of an illustrative collar of a fluid device for automated fluid transfer. FIG. 5D is a rendering of a cross-sectional view of an aspect of an illustrative collar of a fluid device for automated fluid transfer, wherein the cross-section is a plane between a bottom and a top of the fluid device. FIG. 5E is a rendering of a bottom view of an illustrative collar of a fluid device for automated fluid transfer.

FIG. 5F is a rendering of a perspective view of an illustrative collar of a fluid device for automated fluid transfer. FIG. 5G is a rendering of an illustrative liquid venting tube of a collar of a fluid device for automated fluid transfer. FIG. 5H is an image of an illustrative liquid venting tube of a collar of a fluid device for automated fluid transfer. FIG. 5I is a rendering of a perspective view of an illustrative container of a fluid device for automated fluid transfer. FIG. 5J is a rendering of an illustrative fluid device for automated fluid transfer and a robot grasping feature of a robot of a workcell. FIG. 5K is a rendering of a first connection position of robot engagement features of an illustrative fluid device and clamps of a robot grasping feature of a robot of a workcell. FIG. 5L is a rendering of a second connection position of robot engagement features of an illustrative fluid device and clamps of a robot grasping feature of a robot of a workcell.

FIG. 6 is a flow diagram of an illustrative method for automated fluid transfer.

FIG. 7A, FIG. 7B, FIG. 7C, FIG. 7D, FIG. 7E, and FIG. 7F are flow diagrams of illustrative variations of methods for automated fluid transfer.

FIG. 8 is a flow diagram of an illustrative variation of a method for automated fluid transfer.

FIG. 9 is a flow diagram of another illustrative variation of a method for automated fluid transfer.

FIG. 10 is a rendering of a bottom view of an illustrative collar of a fluid device for automated fluid transfer, where the fluid device includes a ball valve.

FIG. 11A is a rendering of a transparent view of an illustrative ball valve for use with the fluid devices herein. FIG. 11B is an exploded view of the illustrative ball valve of FIG. 11A.

FIG. 12A depicts an illustrative variation of fluid flow through a ball valve of a fluid device when the fluid device is in an inverted orientation. FIG. 12B depicts an illustrative variation of fluid flow through a ball valve of a fluid device when the fluid device is in an upright orientation.

### DETAILED DESCRIPTION

A key limiting factor in cell therapy manufacturing is the absence of automated systems, devices, and methods for performing fluid transfer without contamination and/or introducing human error. While devices, including certain bottle closure devices, integrate tubing within a cap of a centrifuge tube, bottle, flask, and the like to enable aseptic fluid handling, these devices still require human intervention and, to date, have not been integrated into automated cell therapy manufacturing processes at scale.

Accordingly, the present disclosure provides systems, devices, and methods for automated fluid transfer within an automated cell processing system in an effort to minimize sterility barriers that limit the availability of manufactured cell therapies at scale.

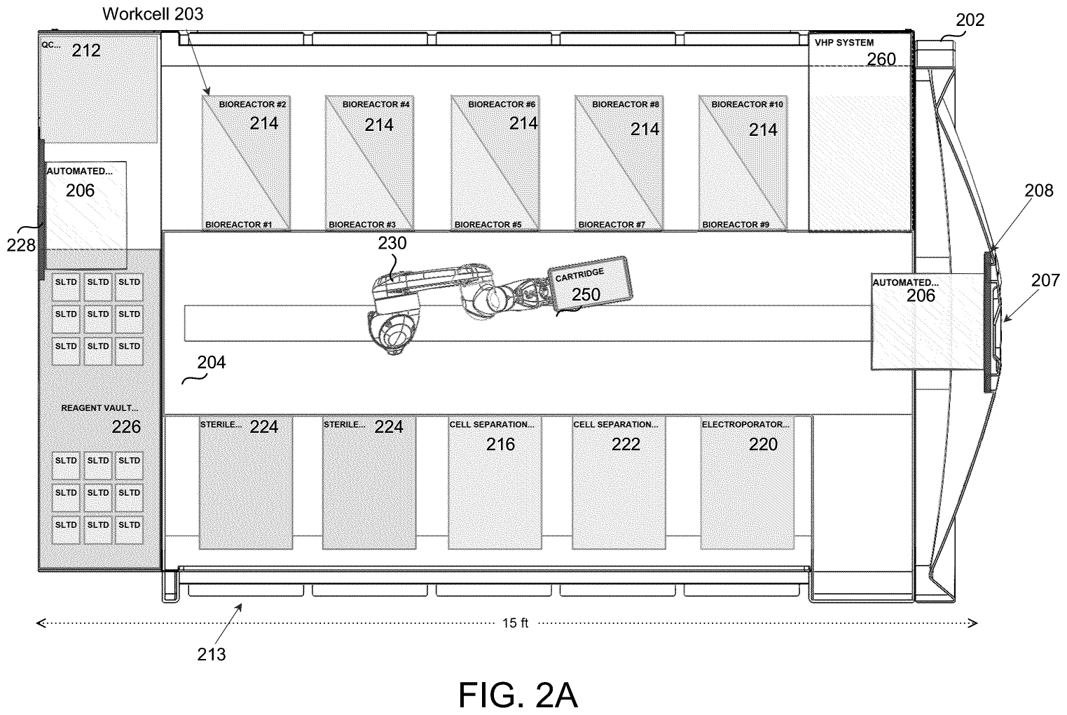

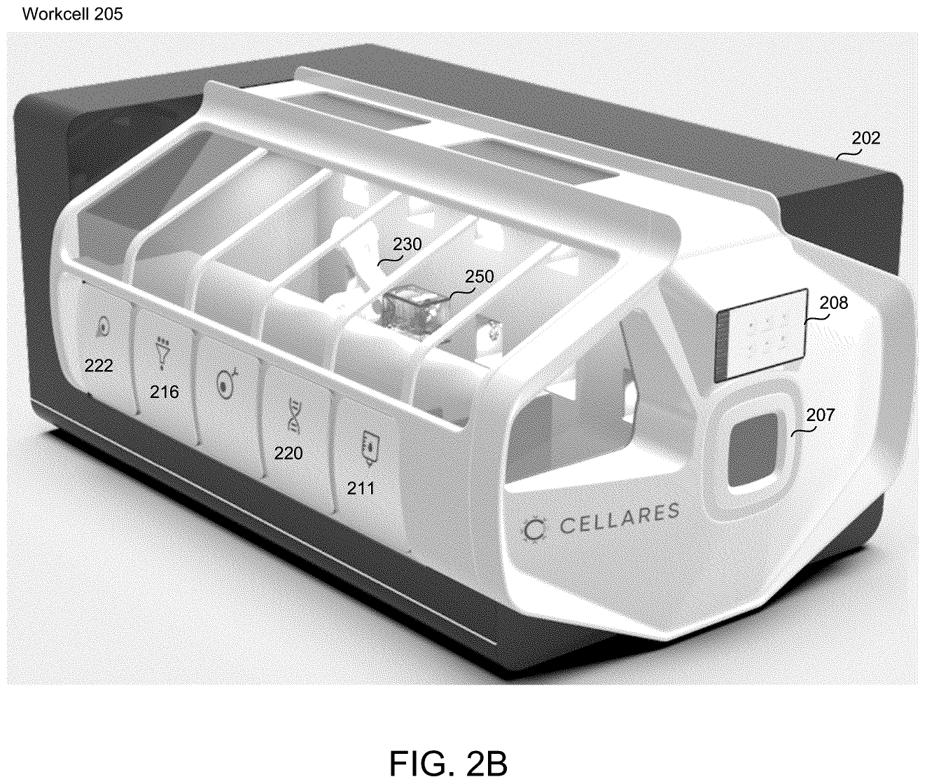

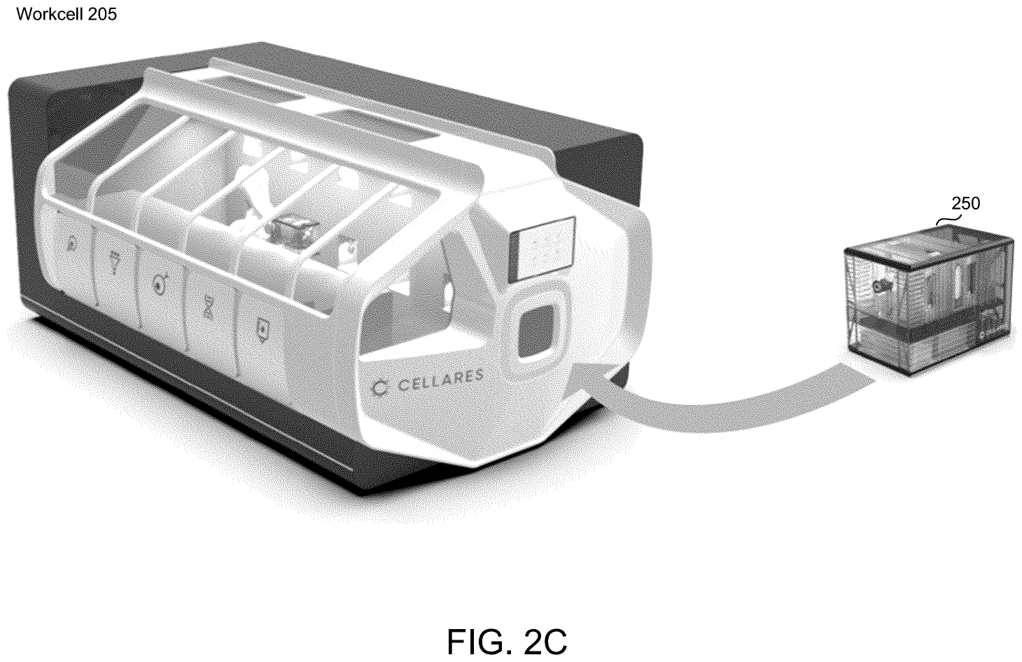

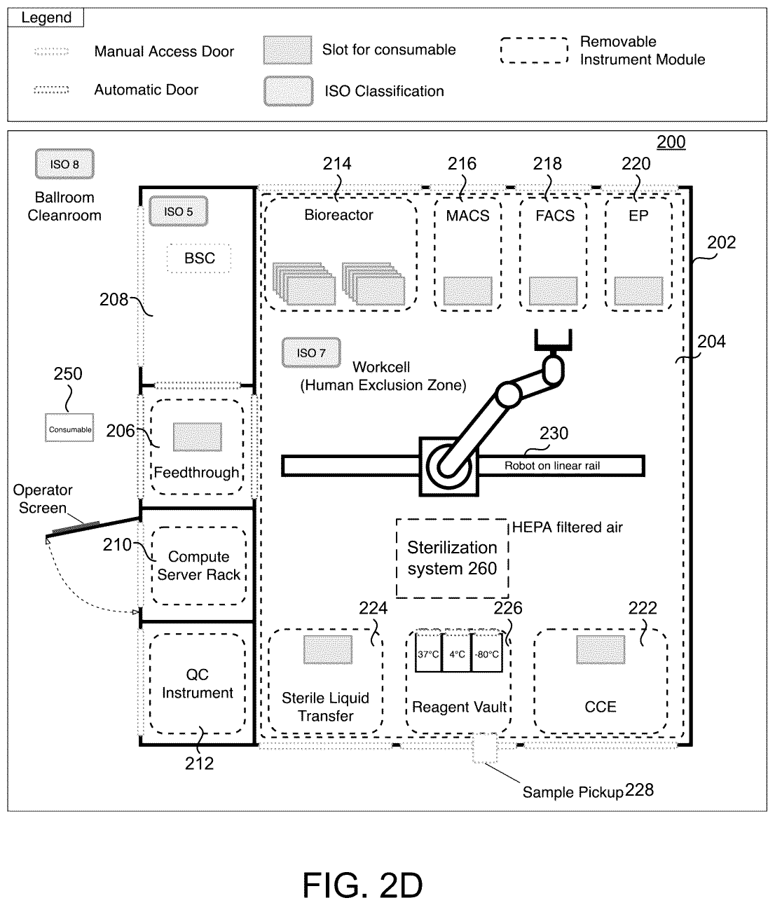

The systems, devices, and methods for performing fluid transfer described herein are for use with a cell therapy manufacturing system, or cell processing system, an exemplary illustration of which is shown in FIG. 2B. As shown in FIG. 2B, cell processing may involve moving a cartridge 250 containing a cell product between a plurality of instruments, such as instruments 211, 216, 220, 222, inside a workcell 202. One or more of the instruments may be configured to couple, engage, or interface with the cartridge 250 to perform cell processing steps on cells within the cartridge 250. In some variations, a plurality of cell processing steps may be performed within the cartridge 250. For example, a robotic arm 230 may be configured to move the cartridge 250 between instruments, each instrument configured to perform a different cell processing step when coupled to a corresponding module within the cartridge 250. In some variations, the cartridge 250 may comprise any number of modules, such as a bioreactor module, a counterflow centrifugal elutriation (CCE) module, a magnetic cell sorter module, an electroporation module, a sorting module (e.g., fluorescence activated cell sorting (FACS) module), an acoustic flow cell module, a microfluidic enrichment module, a spinoculation module, and/or combinations thereof, and the like. In some variations, the workcell 202 may process two or more cartridges in parallel. For example, the workcell 202 may comprise a plurality of instruments, such as instruments 211, 216, 220, 222, having receiving bays. Each instrument may be configured to interface with a cartridge, such as cartridge 250, received within a respective receiving bay, such that multiple instruments within the workcell 202 may be in use at any given time.

Further to and enabling the above cell processing steps, the automated cell processing system may facilitate automated fluid transfers (which may or may not be sterile fluid transfers) between the cartridge and instruments or other components of the system, such as other cartridges and/or sample collection vessels, reagent vessels, waste vessels, other fluid devices, and the like. For example, as will be described below, the systems, devices, and methods of the present disclosure may facilitate fluid transfer between the cartridge and a fluid device, which may be a reagent vessel, a sample collection vessel, a waste vessel, and the like.

### I. Cell Processing System

An illustrative cell processing system for use with the automated fluid transfer devices, systems, and methods is shown in FIG. TA. Shown there is a block diagram of a cell processing system 100 comprising a workcell 110 and controller 120. The workcell 110 may comprise one or more of an instrument 112, a robot 116 (e.g., robotic arm), a reagent vault 118, a sterile liquid transfer port 132, a sterilant source 129, a fluid source 136, a pump 138, and a sensor(s) 151. A cartridge 114 and a fluid device 142, which may be used within the workcell 110 (and so are dashed here). In some variations, the fluid device 142 is a sterile liquid transfer device (SLTD). However, it should be appreciated that the fluid device 142 may be configured to transfer any fluid (which includes liquids), whether sterile or not. In some variations, the fluid device 142 may include a container for storing a fluid (e.g., liquid) and a collar that is couplable to the container and configured to aid in fluid transfer between the fluid device 142 and another component of the system 100 (e.g., the instrument 112 and/or the reagent vault 118). The robot 116 may be configured to move one or more cartridge(s) 114 and fluid device(s) 142 within the workcell 110. As an example, the robot 116 may be configured to move one or more fluid device(s) 142 between the reagent vault 118 one or more instrument(s) 112 (e.g., one or more sterile liquid transfer instruments). In some variations, the robot 118 may be configured to position the fluid device(s) 142 in a first orientation within the reagent vault 118 and in a second orientation (which may be opposite the first orientation) when coupled to the instrument(s) 112. For example, the fluid device(s) 142 may be configured to be stored in an upright orientation within the reagent vault 118, and may be configured to be couplable to the instrument(s) 112 in an inverted position. As is explained herein, the upright orientation may be defined by relative orientations of a collar and a container of a fluid device 142. In some variations, the fluid device 142 may be in the upright orientation when the collar is above the container of the fluid device 142, and may be in the inverted orientation when the container is above the collar of the fluid device 142. The controller 120 may comprise one or more of a processor 122, a memory 124, a communication device 126, an input device 128, and a display 130.

The workcell 110 may comprise a fully, or at least partially, enclosed housing inside which one or more cell processing steps are performed in a fully, or at least partially, automated process. In some variations, the workcell may be an open system lacking an enclosure, which may be configured for use in a clean room, a biosafety cabinet, or other sterile location. The cartridge 114 may be moved using the robot 116 to reduce manual labor in the cell processing steps, and fluid transfers into and out of the cartridge may also be performed in a fully or partially automated process, as will be described in detail herein. For example, one or more fluids may be stored in a fluid device 142. In some variations, the fluid device is able to be moved within the system 100 by the robot 116. The fluid devices and sterile liquid transfer ports described herein advantageously enable the transfer of fluids in an automated and metered manner for automating cell therapy manufacturing.

In some variations, the robot 116 is configured to move cartridges 114 between different instruments to perform a predetermined sequence of cell processing steps. In this way, multiple cartridges 114 may be processed in parallel, as different steps of the cell processing sequence may be performed at the same time on different cartridges.

A sterile liquid transfer port 132 may be coupled between two or more cartridges 114 to transfer a cell product and/or fluid between the cartridges 114. Furthermore, a sterile liquid transfer port 132 may be coupled between any set of fluid-carrying components of the system 100 (e.g., cartridge 114, reagent vault 118, fluid source 136, fluid device 142, etc.). For example, a first sterile liquid transfer port may be coupled between a first cartridge and a corresponding sterile liquid transfer port of a fluid device.

In some variations, a reagent vault 118 (or reagent vaults) is used to store reagents, including but not limited to cell culture media, buffer, cytokines, proteins, enzymes, polynucleotides, transfection reagents, non-viral vectors, viral vectors, antibiotics, nutrients, cryoprotectants, solvents, cellular materials, and pharmaceutically acceptable excipients. Additionally, or alternatively, waste may be stored in the reagent vault, or within a fluid device within the reagent vault. In some variations, in-process samples extracted from one or more cartridges may be stored in the reagent vault, or in a fluid device within the reagent vault. The reagent vault may comprise one or more controlled temperature compartments (e.g., freezers, coolers, water baths, warming chambers, or others, at e.g., about −80° C., about −20° C., about 4° C., about 25° C., about 30° C., about 37° C., and about 42° C.). Temperatures in these compartments may be varied during the cell manufacturing process to heat or cool reagents.

In some variations, the reagents, waste, and/or extracted in-process samples, among others, may be stored within fluid devices 142 within the reagent vault 118. To this end, the fluid devices 142 may be transferred to a cartridge within the workcell or a cartridge may be moved by the robot 116 (or manually by an operator) to the reagent vault 118. The reagent vault 118 can interface with one or more sterile liquid transfer ports on the cartridge, and the reagent or material may be transferred from a fluid device 142 within the reagent vault into the cartridge. Optionally, fluid is added or removed from the cartridge before, during, or after addition or removal of the reagent or material. In some variations, the instruments 112 of the workcell 110 comprise a sterile liquid transfer instrument, similarly configured to transfer fluid into or out of the cartridge in an automated fashion. The sterile liquid transfer instrument may be stocked with reagents by, for example, a robot 116 that moves fluid devices 142 comprising the reagents from a workcell feedthrough or other location to the sterile liquid transfer instrument. In some variations, the robot 116 moves a fluid device(s) 142 from the reagent vault 118 to the sterile liquid transfer instrument. The reagent vault 118 may have automated doors to permit access by the robot 116 to a fluid device(s) 142 stored therein. The fluid device(s) 142 may be configured for pick-and-place movement by the robot 116. In some variations, the reagent vault 118 may comprise one or more sample pickup areas. For example, the robot 116 may be configured to move one or more fluid devices 142 comprising reagents to and from one or more of the sample pickup areas.

In some variations, the sensor(s) 151 of the workcell 110 comprise optical sensors proximate to aspects of a sterile liquid transfer instrument. The sensor(s) 151 may be queried during an automated fluid transfer procedure to aid in the controlled flow of fluids from the fluid device to another fluid device or to a cartridge. In particular, the optical sensors can be arranged with a view to windows of the fluid device to detect the presence or absence of fluid within fluid conduits of the fluid device. In this way, the controller 120 can deliver metered amounts of fluid from the fluid device to an adjoined fluid device or cartridge.

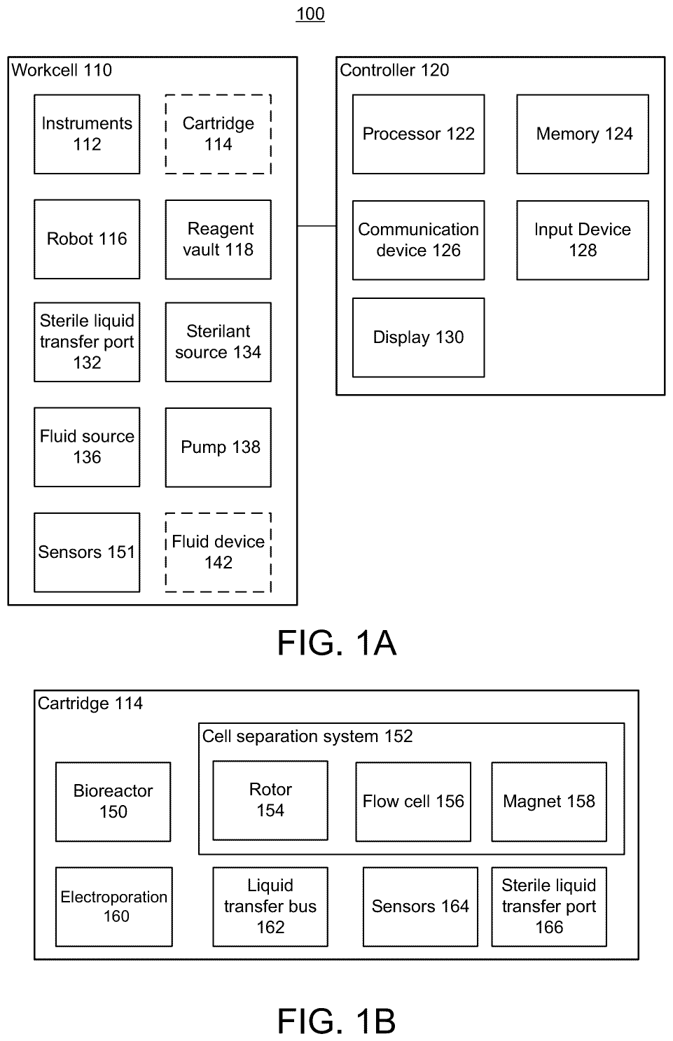

As illustrated in FIG. 1B, a cartridge 114 may comprise one or more of a bioreactor 150, a cell separation system 152, an electroporation module 160, a fluid transfer bus 162, a sensor(s) 164, and a sterile liquid transfer port 166, as described in more detail herein. A cell separation system 152 may comprise one or more of a rotor 154, flow cell 156, and magnet 158. In some embodiments, the magnet 158 may comprise one or more magnets and/or magnet arrays. For example, the cell separation system 152 may comprise a first magnet configured to magnetically rotate a rotor 154 and a second magnet (e.g., magnet array) configured to magnetically separate cells in flow cell 156.

Any suitable cell processing may be performed using the systems and devices described herein, and may include steps such as growing, enriching, selecting, sorting, expanding, activating, transducing, electroporating, washing, and the like. In some variations, a method of processing a solution containing a cell product includes the steps of digesting tissue using an enzyme reagent to release a select cell population into solution, enriching cells using a CCE instrument, washing cells using the CCE instrument, selecting cells in the solution using a selection instrument, sorting cells in the solution using a sorting instrument, differentiating or expanding the cells in a bioreactor, activating cells using an activating reagent, electroporating cells, transducing cells using a vector, and finishing a cell product.

FIG. 2A shows an illustrative cell processing system for use with the devices, systems, and methods described herein. Shown there is workcell 203. The workcell may be divided into an interior zone 204 with a feedthrough 206 access, and quality control (QC) instrumentation 212.

An air filtration inlet (not shown) may provide high-efficiency particulate air (HEPA) filtration to provide ISO7 or better air quality in the interior zone 204. This air filtration may maintain sterile cell processing in an ISO8 or ISO9 manufacturing environment. The workcell 203 may also have an air filter on the air outlet to preserve the ISO rating of the room. Similar to the workcell described above in reference to FIG. 1A, the workcell 203 may further comprise, inside the interior zone 104, a bioreactor instrument 214, a cell selection instrument 216 (e.g., a magnetic separation instrument), an electroporation instrument 220, a counterflow centrifugation elutriation (CCE) instrument 222, a sterile liquid transfer instrument 224 (e.g., for facilitating automated fluid transfers), a reagent vault 226, and a sterilization system 260. The reagent vault 226 may be accessible by a user through a sample pickup port 228. A robot 230 (e.g., support arm, robotic arm, etc.) may be configured to move one or more cartridges 250 from any instrument to any other instrument, move one or more cartridges 250 to and from the reagent vault 226, and/or move one or more fluid devices between the reagent vault 226 and the sterile liquid transfer instrument 224. In some embodiments, the workcell 203 may comprise one or more moveable barriers 213 (e.g., access, door) configured to facilitate access to one or more of the instruments in the workcell 203. FIG. 2B is a perspective view of a workcell 205 of a cell processing system. FIG. 2C is a perspective view of a cell processing system depicting a cartridge 250 introduced into a workcell 205. A plurality of cartridges may be inserted into the workcell 205 and undergo one or more cell processing operations in parallel.

FIG. 2D is a schematic illustration of an embodiment of a workcell 200. Workcell 200 may comprise an enclosure 202 having four walls, a base, and a roof. The workcell 200 may be divided into an interior zone 204 with a feedthrough 206 access, a biosafety cabinet (BSC) 208, compute server rack 210 (e.g., controller 120), and quality control (QC) instrumentation 212. An air filtration inlet (not shown) may provide high-efficiency particulate air (HEPA) filtration to provide ISO7 or better air quality in the interior zone 204. The workcell 200 may also have an air filter on the air outlet to preserve the ISO rating of the room. As with the workcell described above, workcell 200 may further comprise, inside the interior zone 204, an instrument 211 (e.g., disposed in a universal instrument bay), a bioreactor instrument 214, a cell selection instrument 216 (e.g., magnetic separation instrument, cell selection system), a cell sorting instrument 218 (e.g., FACS), an electroporation instrument 220, a counterflow centrifugation elutriation (CCE) instrument 222, and a sterile liquid transfer instrument 224, alongside a reagent vault 226 and a sterilization system 260 comprising one or more of a sterilant source, fluid source, and a pump. As will be described below, the sterilization system 260 may be connectable to a fluid device for sterilization of a sterile liquid transfer port during an automated fluid transfer process. The reagent vault 226 may be accessible through a sample pickup port 228. A robot 230 (e.g., support arm, robotic arm) may be configured to move one or more cartridges 250 from any instrument to any other instrument, move one or more cartridges 250 to and from the reagent vault 226, and/or move one or more fluid devices between the reagent vault 226 and the sterile liquid transfer instrument 224.

In some embodiments, a human operator may load one or more cartridges 250 into the feedthrough 206. The cartridges 250 may be pre-sterilized, or the feedthrough 206 may sterilize the cartridge 250 using ultraviolet radiation (UV) or chemical sterilizing agents provided as a spray or wash. The feedthrough 206 chamber may optionally be configured to automatically spray, wash, irradiate, or otherwise treat cartridges (e.g., with ethanol and/or isopropyl alcohol solutions) to maintain sterility of the interior zone 204 (e.g., ISO 7 or better) or the biosafety cabinet 208 (e.g., ISO 5 or better). The cartridge 250 may be passed to the biosafety cabinet 208, where input cell product is provided and loaded to the cartridge 250. The user may then move the cartridge 250 back to the feedthrough 206 and initiate automated cell processing using a computer processor in the computer server rack 210 (e.g., controller 120). The robot 230 may be configured to move the cartridge 250 in a predefined sequence to a plurality of instruments and stations, with the components of the workcell 200 being controlled by the computer processor of the computer server rack 210.

Other suitable cell processing systems and aspects thereof are provided e.g., in U.S. patent application Ser. No. 17/198,134, published as U.S. Patent Publication No. 2021/0283565, entitled “Systems and Methods for Cell Processing”, which is incorporated by reference herein.

### A. Workcell

i. Robot

Generally, a robot of the workcell may comprise any mechanical device capable of moving a cartridge and/or a fluid device from one location to another location within the workcell. For example, the robot may comprise a mechanical manipulator (e.g., an arm) in a fixed location, or attached to a linear rail, or a 2- or 3-dimensional rail system. While shown in some of the Figures as being fixed in place or with respect to a rail system, the robot need not be so. For example, in some variations, the robot comprises a wheeled device. Any number of robots may be used within the workcell, as described herein. For example, in some embodiments, the workcell comprises two or more robots of the same or different type (e.g., two robotic arms each independently configured for moving cartridges between instruments). The robot may also comprise an end effector for precise handling of different cartridges or fluid devices or for barcode scanning or radio-frequency identification tag (RFID) reading.

The robots for use with the cell processing systems described herein are capable of moving cartridges between slots or bays in the workcell so that the modules within the cartridge can couple to corresponding instruments within the workcell to perform different cell processing steps. Further, the robots for use with the cell processing systems described herein are capable of moving and manipulating fluid devices within the workcell. For instance, the robot may be capable of moving a reagent storing fluid device from a reagent vault of the workcell to a sterile liquid transfer instrument of the workcell so that automated fluid transfer between the reagent storing fluid device and a cartridge can be performed.

ii. Controller

In embodiments, a cell processing system 100 may comprise a controller 120 (e.g., computing device) comprising one or more of a processor 122, memory 124, communication device, 126, input device 128, and display 130. The controller 120 may be configured to control (e.g., operate) the workcell 110. The controller 120 may comprise a plurality of devices. For example, the workcell 110 may enclose one or more components of the controller 120 (e.g., processor 122, memory 124, communication device 126) while one or more components of the controller 120 may be provided remotely to the workcell 110 (e.g., input device 128, display 130).

iii. Processor

The processor (e.g., processor 122) described here may process data and/or other signals to control one or more components of the system. The processor may be configured to receive, process, compile, compute, store, access, read, write, and/or transmit data and/or other signals. Additionally, or alternatively, the processor may be configured to control one or more components of a device (e.g., console, touchscreen, personal computer, laptop, tablet, server).

In some embodiments, the processor may be configured to access or receive data and/or other signals from one or more of workcell 110, server, controller 120, and a storage medium (e.g., memory, flash drive, memory card, database). In some embodiments, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data transfer), and/or central processing units (CPU). The processor may be, for example, a general-purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC).

Software modules (executed on hardware) may be expressed in a variety of software languages (e.g., computer code), including structured text, typescript, C, C++, C #, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

iv. Memory

The cell processing systems and devices described here may include a memory (e.g., memory 124) configured to store data and/or information. In some embodiments, the memory may include one or more of a random-access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some embodiments, the memory may store instructions to cause the processor to execute modules, processes, and/or functions associated with the device, such as image processing, image display, sensor data, data and/or signal transmission, data and/or signal reception, and/or communication. Some embodiments described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The computer code (also may be referred to as code or algorithm) may be those designed and constructed for the specific purpose or purposes. In some embodiments, the memory may be configured to store any received data and/or data generated by the controller and/or workcell. In some embodiments, the memory may be configured to store data temporarily or permanently.

v. Input Device

In some embodiments, the input device, for example, input device 128 may comprise or be coupled to a display. Input device may be any suitable device that is capable of receiving input from a user, for example, a keyboard, buttons, touch screen, etc. The input device may include at least one switch configured to generate a user input. For example, an input device may include a touch surface for a user to provide input (e.g., finger contact to the touch surface) corresponding to a user input. An input device including a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In embodiments of an input device including at least one switch, a switch may have, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a user input. A microphone may receive audio data and recognize a user voice as a user input.

In some embodiments, the cell processing system may optionally include one or more output devices in addition to the display, such as, for example, an audio device and haptic device. An audio device may audibly output any system data, alarms, and/or notifications. For example, the audio device may output an audible alarm when a malfunction is detected. In some embodiments, an audio device may include at least one of a speaker, a piezoelectric audio device, a magnetostrictive speaker, and/or a digital speaker. In some embodiments, a user may communicate with other users using the audio device and a communication channel. For example, a user may form an audio communication channel (e.g., VoIP call).

vi. Communication Device

In some embodiments, the controller may include a communication device (e.g., communication device 126) configured to communicate with another controller and one or more databases. The communication device may be configured to connect the controller to another system (e.g., Internet, remote server, database, workcell) by wired or wireless connection. In some embodiments, the system may be in communication with other devices via one or more wired and/or wireless networks. In some embodiments, the communication device may include a radiofrequency receiver, transmitter, and/or optical (e.g., infrared) receiver and transmitter configured to communicate with one or more devices and/or networks. The communication device may communicate by wires and/or wirelessly.

vii. Display

Image data may be output on a display e.g., display 130) of a cell processing system. In some embodiments, a display may include at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

viii. Graphical User Interface

In some embodiments, as indicated above, a GUI may be configured for designing a process and monitoring a product. For example, the GUI may be a process design home page. The GUI may indicate that no processes have been selected or loaded. A create icon (e.g., “Create a Process”) may be selectable for a user to begin a process design process. In some embodiments, one or more of the GUIs described herein may include a search bar.

### B. Cartridge

The cell processing systems described herein may comprise one or more cartridges having one or more modules configured to interface with one or more instruments within the workcell.

An exemplary cartridge was described with reference to FIG. 1B.

Various materials may be used to construct the cartridge and the cartridge housing, including metal, plastic, rubber, and/or glass, or combinations thereof. The cartridge, its components, and its housing may be molded, machined, extruded, 3D printed, or any combination thereof. The cartridge may contain components that are commercially available (e.g., tubing, valves, fittings)—these components may be attached or integrated with custom components or devices. The housing of the cartridge may constitute an additional layer of enclosure that further protects the sterility of the cell product.

In some embodiments, the cartridge modules may be comprised of distinct sections that are integrated in a fixed configuration within the cartridge. Additionally, or alternatively, the modules may be configurable or moveable within the cartridge, permitting various formats of cartridges to be assembled. For example, the cartridge can be a single, closed unit with fixed components for each module, or the cartridge may contain configurable modules coupled by configurable fluidic, mechanical, optical, and electrical connections. In some variations, one or more sub-cartridges, each containing a set of modules, may be used to perform various cell processing workflows. The modules may each be provided in a distinct housing or may be integrated into a cartridge or sub-cartridge with other modules. The disclosure generally shows modules as distinct groups of components for the sake of simplicity, but it should be noted that these modules may be arranged in any suitable configuration. For example, the components for different modules may be interspersed with each other such that each module is defined by the set of connected components that collectively perform a predetermined function. However, the components of each module may or may not be physically grouped within the cartridge. In some embodiments, multiple cartridges may be used to process a single cell product through transfer of the cell product from one cartridge to another cartridge of the same or different type and/or by splitting cell product into more cartridges and/or pooling multiple cell products into fewer cartridges.

Generally, each of the instruments within the workcell interfaces with its respective module or modules on the cartridge. For example, when a cartridge has an electroporation module, it is moved by the robot to the electroporation instrument within the workcell to perform electroporation on the cells within the cartridge. One advantage of such split module/instrument designs is that expensive components (e.g., motors, sensors, heaters, lasers, etc.) may be retained in the instruments of the system while less expensive components reside in the cartridge, which can be configured for single-use. The use of disposable cartridges may eliminate the need to sterilize cartridges between use. Furthermore, having multiple instruments within the workcell further helps allow for the parallel utilization of those instruments when multiple cartridges are used within the workcell. In contrast, most conventional semi-automated instruments have instrument components that sit idle and are incapable of simultaneous parallel use.

In some embodiments, the cartridge comprises a sterile liquid transfer port for fluid transfer into and out of the cartridge. In some variations, the cartridge comprises any number of sterile liquid transfer ports and any number or position of fluid paths between modules and the sterile liquid transfer ports.

The sterile liquid transfer ports described herein may form a sterile fluid pathway between a fluid device and a cartridge and/or a first cartridge and a second cartridge to enable fluid transfer that may be sterile, fully automated, and precisely metered (e.g., precise control of a transferred fluid volume). In some variations, the robot may be configured to operate the sterile liquid transfer port to open and close a set of ports and valves thereof to permit fluid flow between a fluid device and a cartridge and/or a first cartridge and a second cartridge. The use of a robot and controller to operate the sterile liquid transfer port may facilitate automation and sterility of a cell processing system.

Additional aspects of suitable cartridges are provided e.g., in U.S. patent application Ser. No. 17/198,134, published as U.S. Patent Publication No. 2021/0283565, entitled “Systems and Methods for Cell Processing”, which is incorporated by reference herein.

### C. Fluid Device and System for Automated Fluid Transfer

Generally, the fluid devices described herein may be configured to store fluid for automated transfer to another component of a cell processing system, such as a cartridge, an instrument, another fluid device, or the like. In some variations, the fluid device for automated fluid transfer may comprise a portable consumable configured to be moved and manipulated within a workcell using a robot. For example, a robot may be configured to move a fluid device within the workcell from a reagent vault to an ISO 7 space to a sterile liquid transfer instrument within a cell processing system, such as that described above. The fluid device may enable the transfer of fluids in an automated, sterile, and metered manner for automating cell therapy manufacturing.

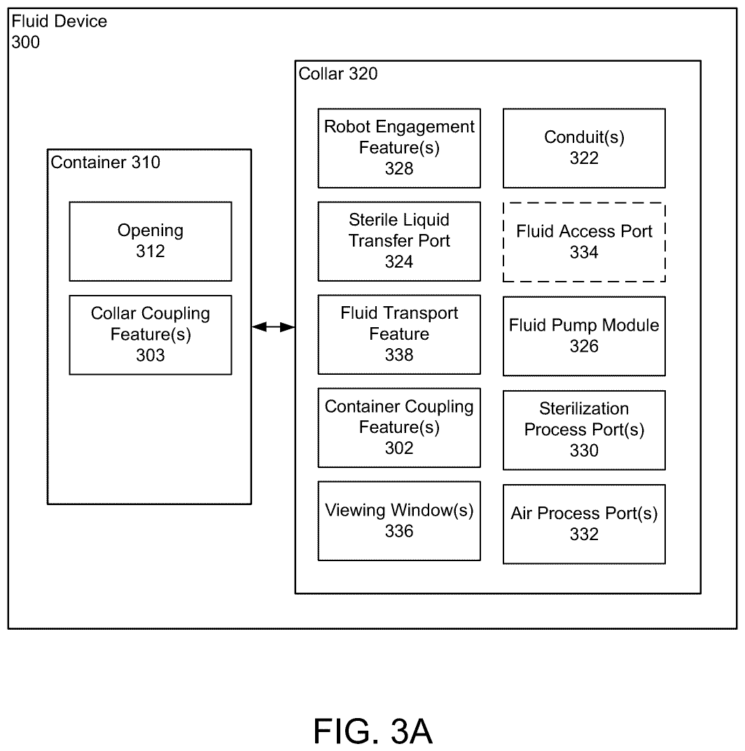

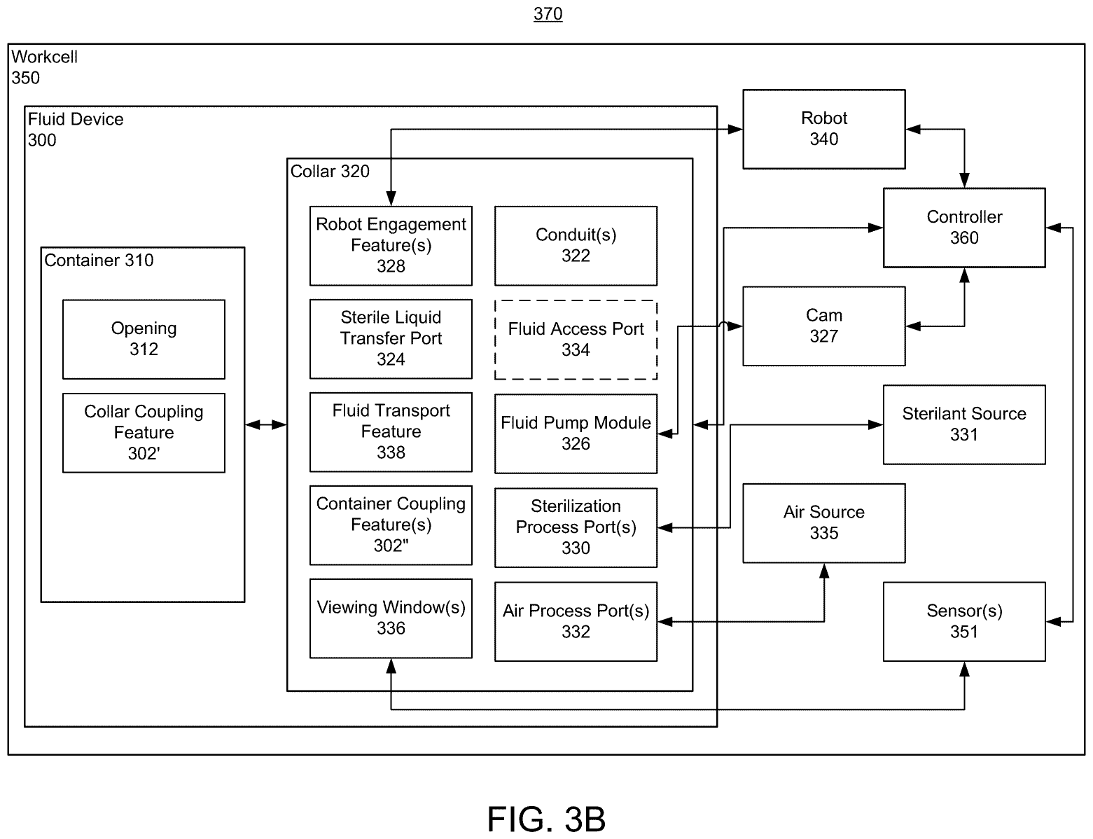

Turning now to FIG. 3A, a schematic diagram of an illustrative variation of a fluid device 300 is shown. In some embodiments, the fluid device 300 comprises a container 310 and a collar 320. The container 310 may comprise an opening 312 and at least one collar coupling feature 303. In some variations, the opening 312 of the container 310 comprises an annular seal. The collar 320 may comprise one or more robot engagement features 328, fluid conduits 322, a sterile liquid transfer port 324, a fluid transport feature 338 couplable to the opening 312 of the container 310, at least one container coupling feature 302 couplable to a corresponding one of the at least one collar coupling feature 303 of the container 310, at least one viewing window 336, a fluid pump module 326, a plurality of ports including sterilization process port(s) 330 and at least one air process port 332, and, optionally, a fluid access port 334. In some embodiments, the annular seal of the opening 312 of the container 310 contacts the fluid transport feature 338 when the container 310 is coupled to the collar 320 and prevents leakage and/or contamination via the coupling.

In some embodiments, as introduced above, the container coupling feature 302 is releasably couplable to the collar coupling feature 303 of the container 310. The container coupling feature 302 and the collar coupling feature 303 may be universally designed such that containers are interchangeably couplable to the collar 320. In this way, a collar 320 can be used with any size and shape container 310. In some variations, a range of containers capable of holding a range of fluid volumes can be used. For example, container 310 may be capable of holding between about 1 ml and about 1 L, or at least about 1 ml, at least about 2 ml, at least about 3 ml, at least about 4 ml, at least about 5 ml, at least about 10 ml, at least about 15 ml, at least about 20 ml, at least about 25 ml, at least about 50 ml, at least about 100 ml, at least about 200 ml, at least about 250 ml, at least about 500 ml, and/or at least about 750 ml. In some variations, the opening 312 of the container may be couplable to a fluid transport feature 338 of the collar 320. To this end, the opening 312 and the fluid transport feature 338 may be couplable by a threaded interface, a compression fit, a press fit, a friction fit, a luer fit, or couplable by another suitable coupling method that permits fluid transfer between the container 310 and the collar 320 without fluid leaks and/or contamination.

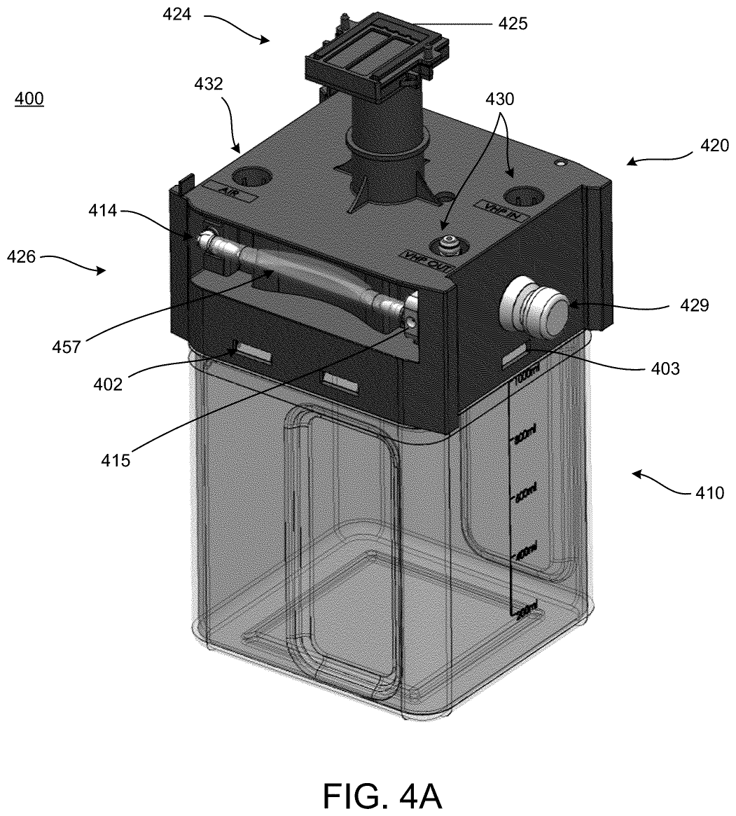

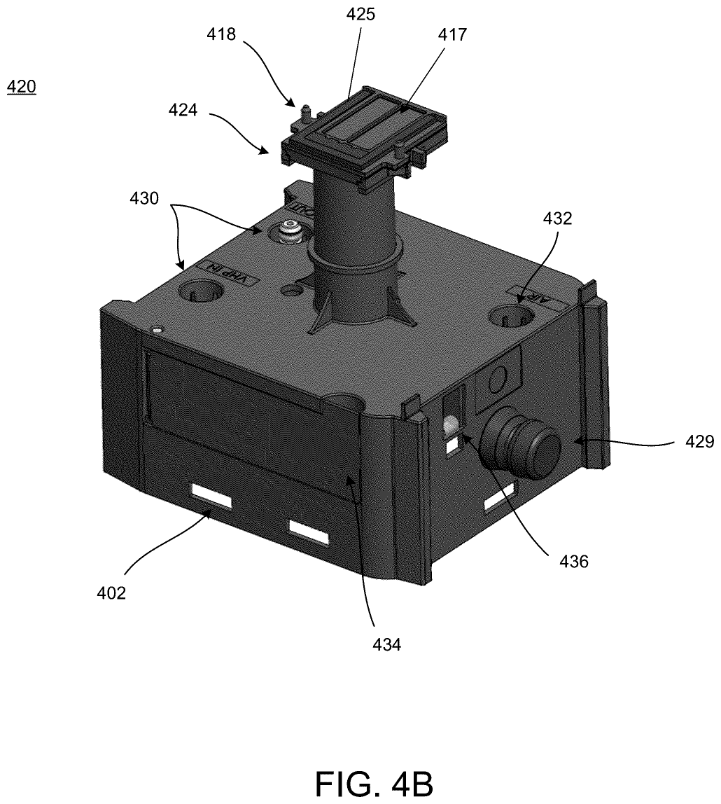

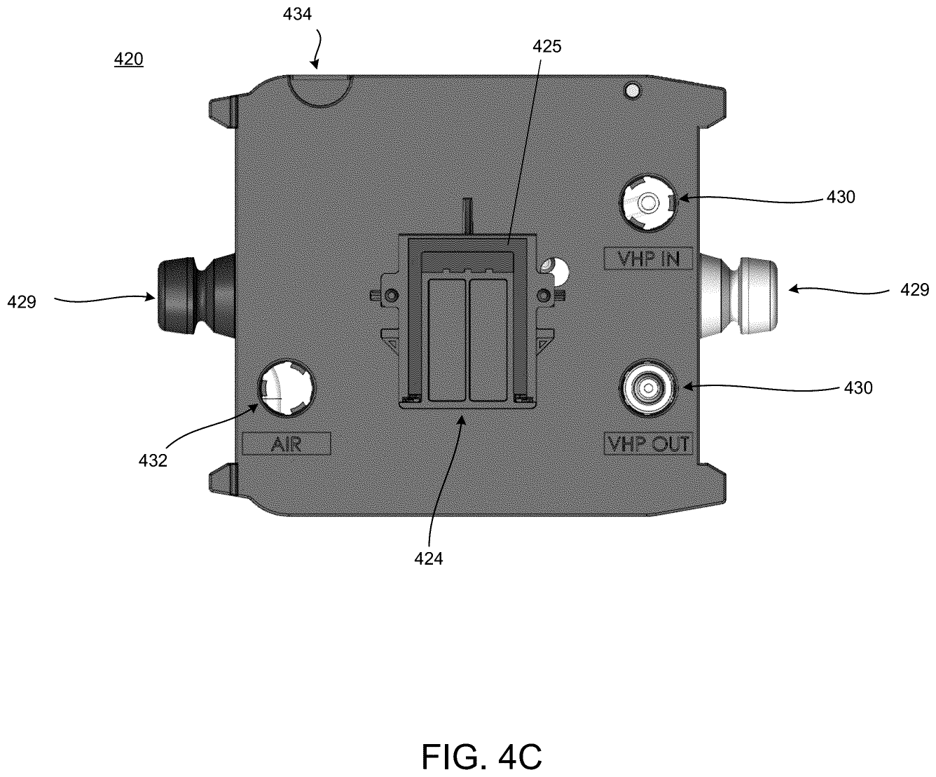

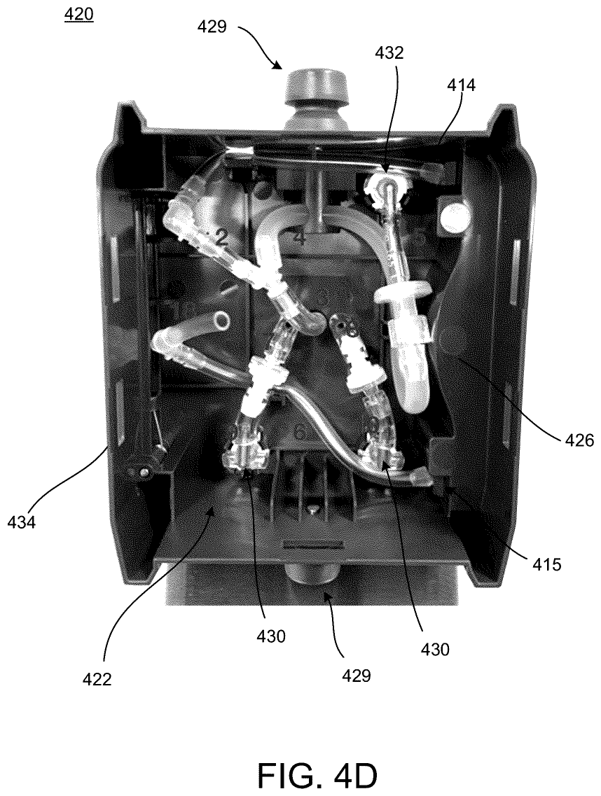

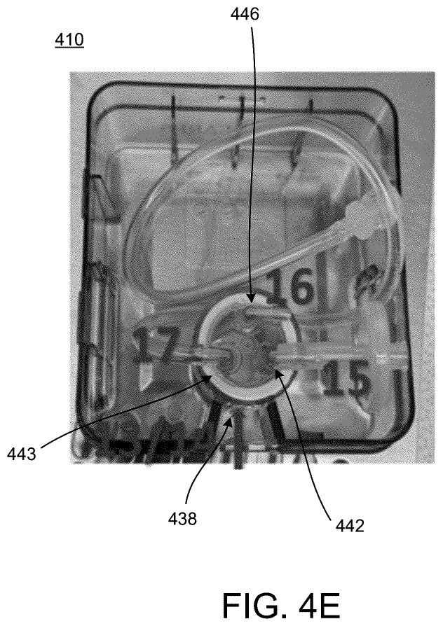

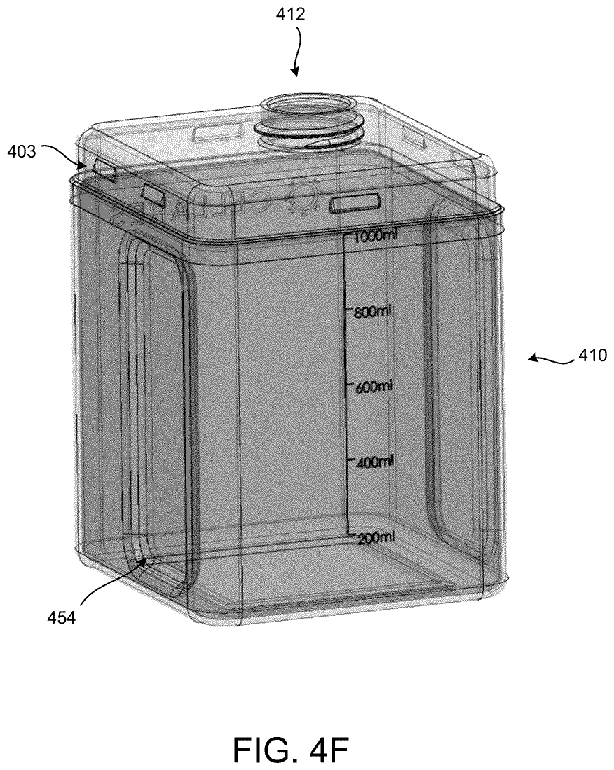

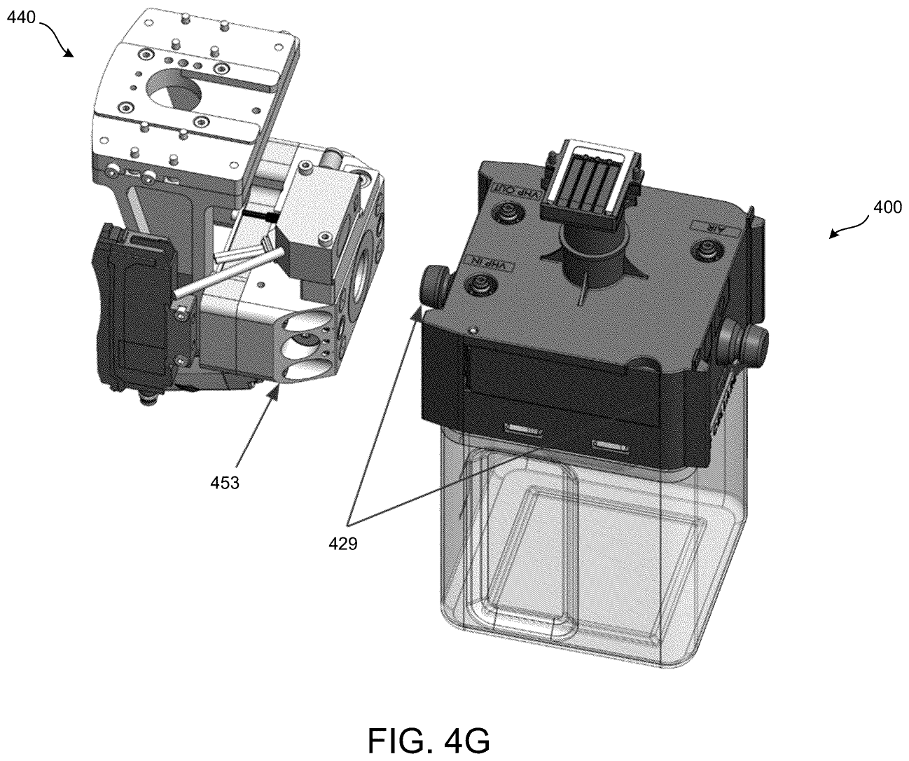

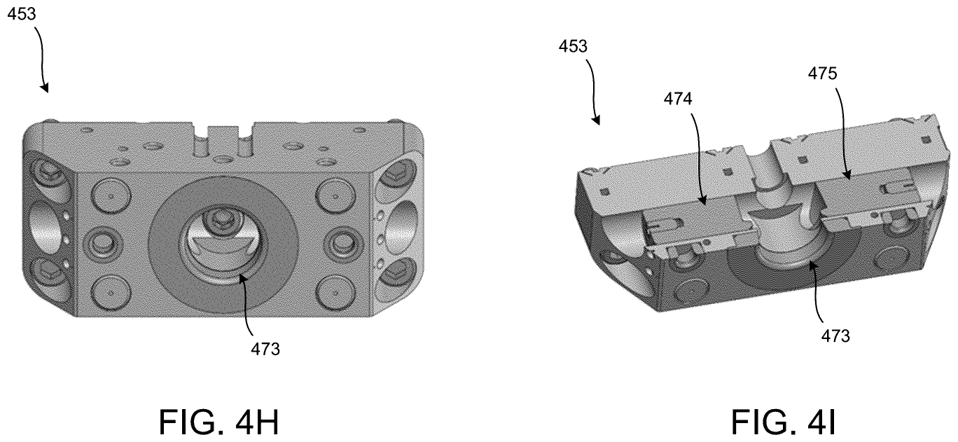

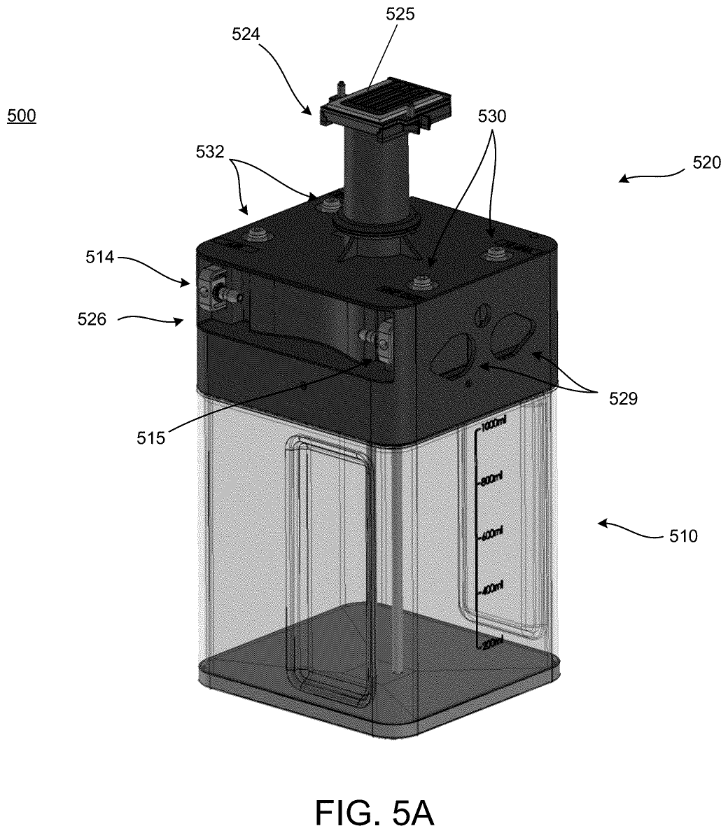

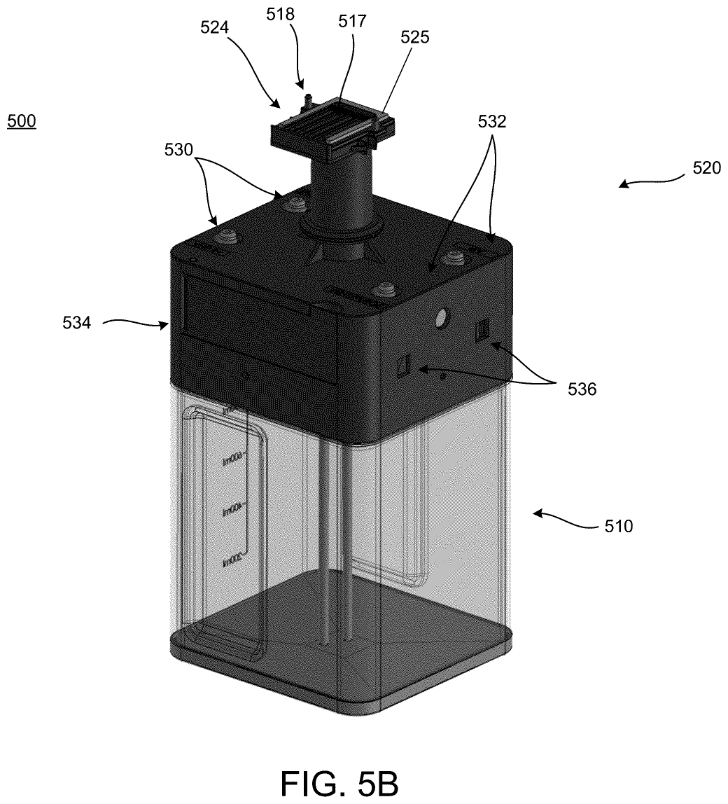

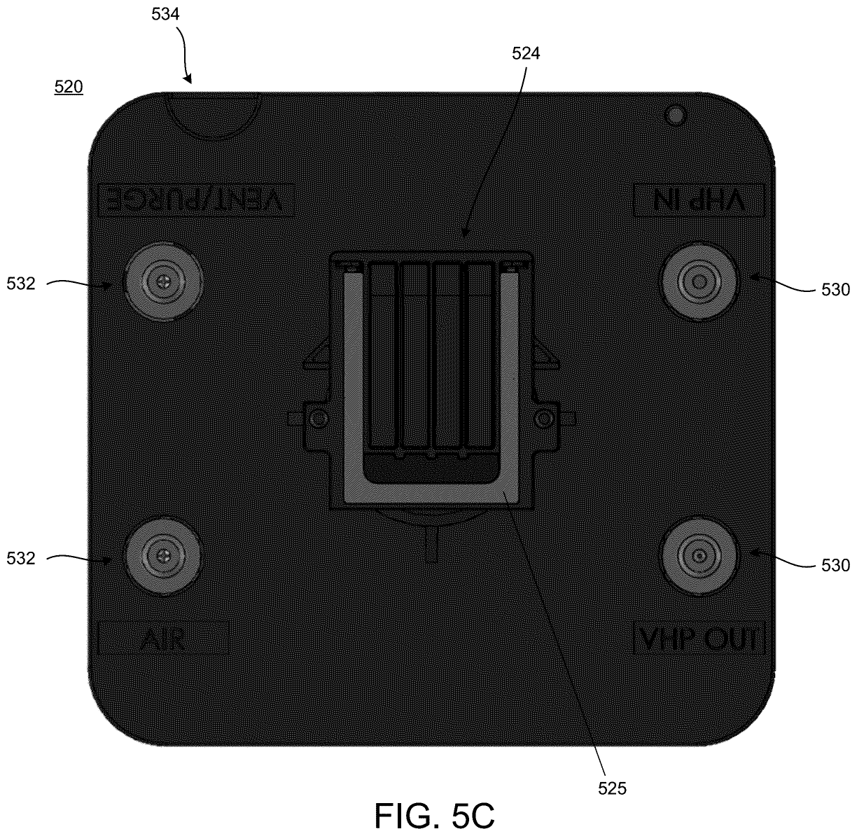

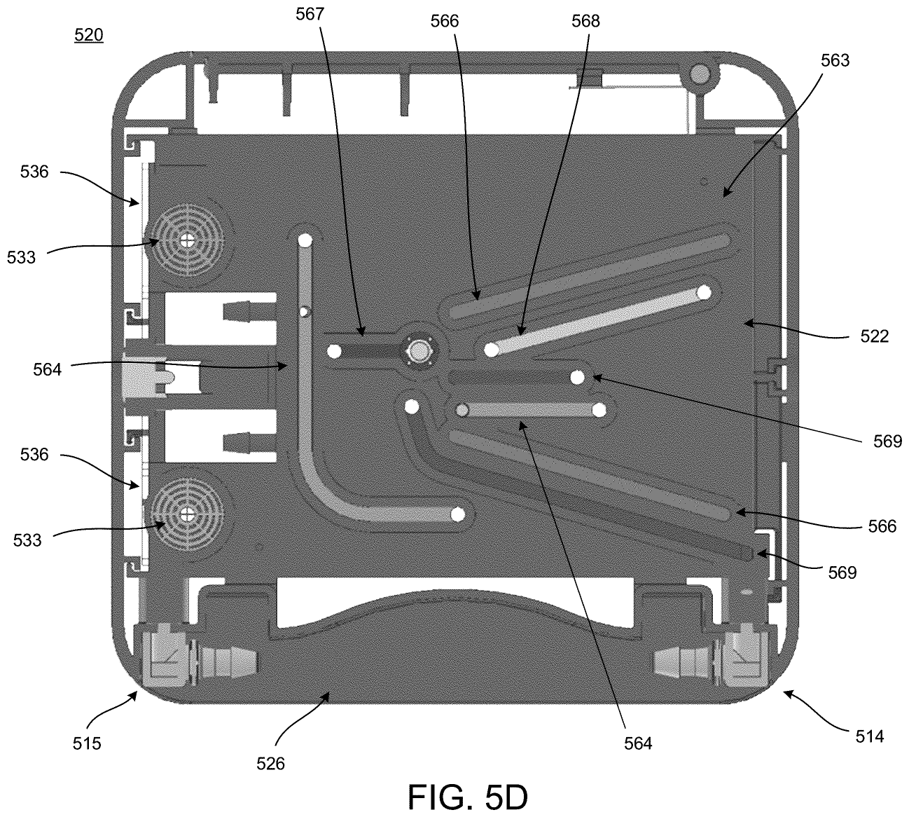

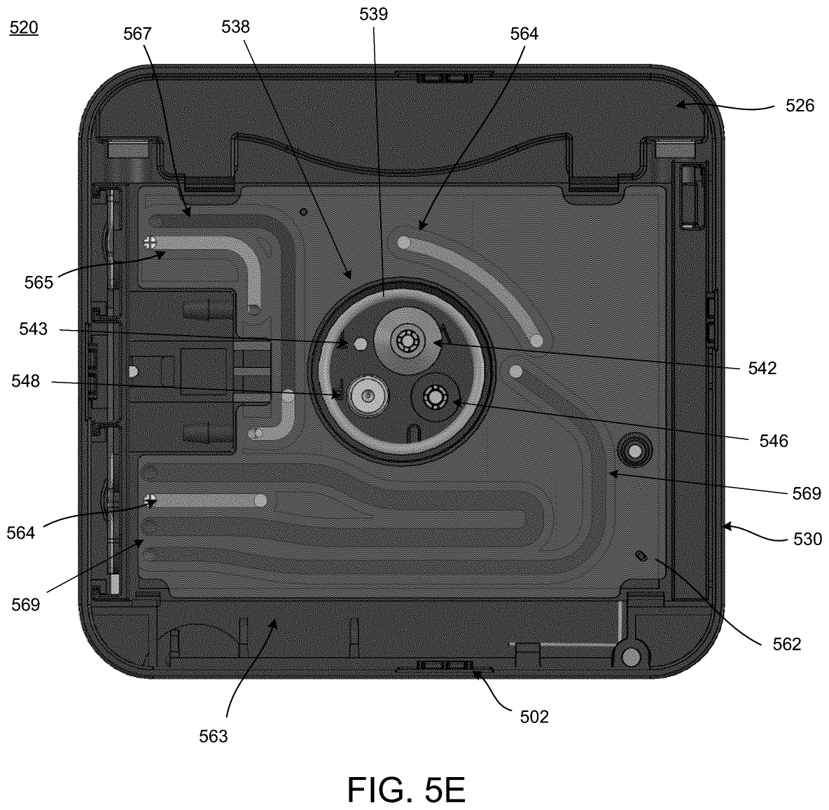

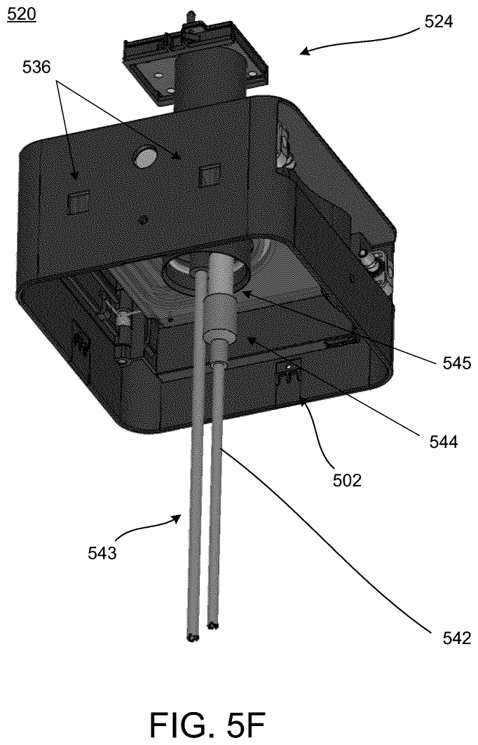

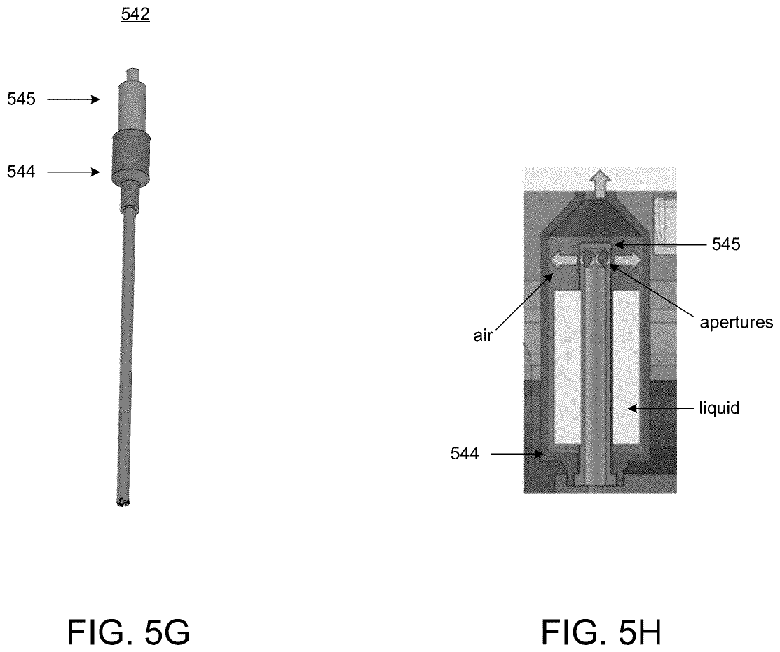

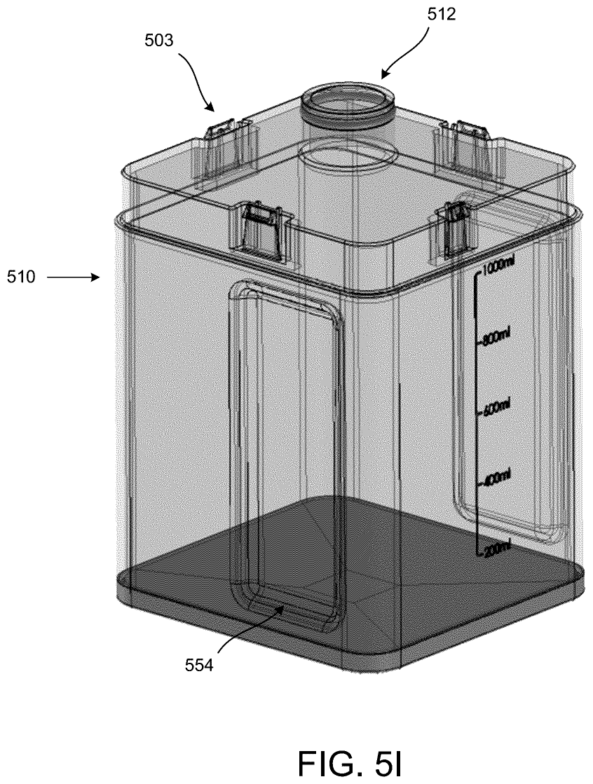

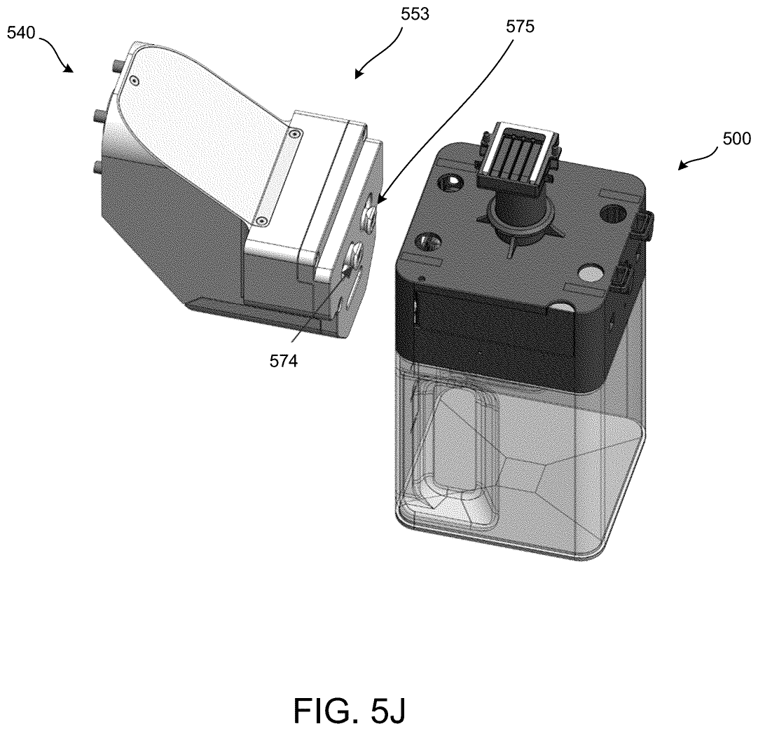

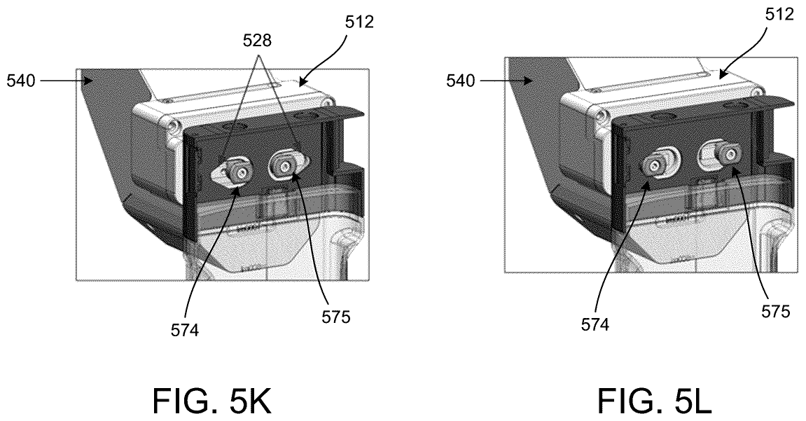

In some embodiments, the one or more robot engagement features 328 of the collar 320 may be engageable by a robot of a workcell to move and otherwise manipulate the fluid device 300 within the workcell. This allows for automated pick and place of the fluid device 300 within the workcell. Manipulation of the fluid device 300 within the workcell is described in more detail with reference to FIG. 4G through FIG. 4I and FIG. 5J through FIG. 5L. In some variations, the one or more robot engagement features 328 may be at least one depression and/or protrusion within or on a surface of the collar 320. In some embodiments, the one or more robot engagement features 328 of the collar 320 may also be configured or configurable to permit different storage orientations with a reagent vault of the workcell. For instance, the one or more robot engagement features 328 of the collar 320 may be configured to allow for hanging the fluid device 300 in e.g., an inverted orientation with the reagent vault.

In some embodiments, the fluid pump module 326 of the collar 320 may be a fluid pump configured to move fluid through the fluid conduits 322 of the collar 320. For instance, the fluid pump may be a centrifugal pump or a positive-displacement pump. In some variations, the fluid pump module 326 may comprise compressible fluidic tubing exposed to an external environment of the collar 320. The compressible fluidic tubing may be coupled between an outlet port of the collar 320 which delivers fluid to the compressible fluidic tubing and an inlet port of the collar 320 which returns the fluid to the fluid conduits 322 of the collar 320. In particular, the compressible fluidic tubing may be proximate an external surface of the collar 320 so that an affector of the workcell may interact with the compressible fluidic tubing to move fluid therein. For example, the compressible fluidic tubing of the collar 320 and the affector of the workcell may constitute a peristaltic pump. After coupling of the fluid device 300 to, for instance, a cartridge, the affector, which may be a cam mechanism (or the like), within the workcell, can be iteratively contacted against the compressible fluidic tubing. Iterative contact between the affector and the compressible fluidic tubing results in iterative, controllable compression of the compressible fluidic tubing. Based on a direction of movement of the affector, this iterative compression pushes fluid toward the inlet port of the collar 320 and pulls fluid away from the outlet port of the collar 320, ultimately resulting in the transfer of fluid between the fluid device 300 and the cartridge. It can be appreciated, however, that the affector may be operated in other manners as well. For example, it may be operated to permit bidirectional movement of fluids within the compressible fluidic tubing and, thus, the fluid conduits 322 of the collar 320. This bidirectional movement of fluids allows for the possibility of flowing fluids out of the fluid device 300, such as for culture medium replenishment, and also flowing fluids into the fluid device 300, such as for sample collection.

In some variations, the at least one viewing window 336 can be an aperture, region of translucency and/or transparency, or any other type of viewpoint providing a vantage to at least a segment of the fluid conduits 322 of the collar 320 and permitting evaluation of fluid movement within the fluid conduits 322. In some variations, the evaluation may be an optical evaluation of the fluid movement within the fluid conduits 322 performed by a sensor(s) disposed on, for instance, a sterile liquid transfer instrument of the workcell. For example, a sensor disposed on the sterile liquid transfer instrument of the workcell may be aligned with the at least one viewing window 336 of the collar 320 and may detect a transition from air to liquid within the fluid conduits 322, thereby indicating the beginning of metered fluid transfer, or may detect a transition from liquid to air within the fluid conduits 322, thereby indicating an emptying of the container 310. In other variations, the evaluation of the fluid movement within the fluid conduits 322 may be performed by an onboard sensor configured to perform optical, thermal, or electromagnetic evaluation of the fluid conduits 322 to determine an air-liquid interface therein.

In some embodiments, the sterile liquid transfer port 324 of the collar 320 may comprise at least one of a port and a valve and may form one part of a sterile fluid pathway between the fluid device 300 and another fluid device and/or cartridge to enable sterile, automated, and precisely metered (e.g., precise control of a transferred fluid volume) fluid transfer. In some variations, the sterile liquid transfer port 324 may further comprise a mechanical seal and/or pathways for sterilant to be delivered within the sterile liquid transfer port 324. In some variations, the mechanical seal may be formed on a surface of or as a component of the port of the sterile fluid transfer port 324. Separately, or together, the mechanical seal and the sterilant pathway help ensure sterility of the fluid transfer pathway. In some variations, as will be described in more detail below, a robot of the workcell, which may be a robot of a sterile liquid transfer instrument, may be configured to operate the sterile liquid transfer port 324 to open and close a set of ports and valves thereof to permit fluid flow between the fluid device 300 and the cartridge and/or the other fluid device. Additional detail regarding sterile liquid transfer ports and aspects thereof are provided e.g., in U.S. patent application Ser. No. 17/331,556, now issued as U.S. Pat. No. 11,376,587, entitled “Fluid Connector”, which is incorporated by reference herein.

In some variations, the sterilization process ports 330 and/or the at least one air process port 332 may comprise valves (e.g., actuatable valves, passive valves). For example, the sterilization process ports 330 may be coupled to passive valves and may deliver sterilant from a sterilant source within the workcell to the fluid conduits 322 and the sterile liquid transfer port 324 of the collar 320. In an example, the sterilant source may contain the sterilant such as vaporized hydrogen peroxide, ionized hydrogen peroxide, chlorine dioxide, ethylene oxide, and the like. As described throughout, the sterilant may be provided to the sterile liquid transfer port 324 via the fluid conduits 322 after coupling of the sterile liquid transfer port 324 to a corresponding sterile liquid transfer port of a cartridge, another fluid device, or the like. In some variations, the sterilant may be provided to the sterile liquid transfer port 324 after corresponding ports of the connected sterile liquid transfer ports are actuated, the actuation also translating the mechanical seals away from the fluid pathway, but before corresponding valves of the connected sterile liquid transfer ports are connected and fluid is pumped from the fluid device 300 to the cartridge, the other fluid device, or the like. In this way, the sterilant may also be provided to the interface between the corresponding valves of the connected sterile liquid transfer ports before fluid is flowed. In some variations, a fluid pump may be coupled to the sterilant source and fluid signals can be generated to control circulation of the sterilant into and out of the sterile liquid transfer port 324 via the sterilization process ports 332. In some variations, the sterilization process ports 330 and/or the at least one air process port 332 may be non-valved. In some variations, the at least one air process port 332 may be valved (e.g., pinch valves) and may provide a pathway for air to enter or leave the fluid device 300 during filling of the container 310 and/or depleting of the container 310. In some variations, the at least one air process port 332 may be connected to an air source. In some variations, the air source may comprise compressed air, which may be used to purge the fluid conduits 322 of the collar 320 before and/or after fluid transfer therethrough. In some variations, the at least one air process port 332 is connected to an in-line filter capable of preventing the introduction of contaminants into the fluid device 300 during filling of the container 310 and/or depleting of the container 310. In some variations, the in-line filter may be a hydrophobic filter.

In some variations, an air process port 332 having a valve (e.g., a pinch valve) may risk leaking fluid (e.g., liquid) from the container 310 when the fluid device 300 is in certain orientations. For example, the fluid device 300 may be configured to couple to one or more components of the workcell 350 in various orientations. A first orientation of the fluid device 300 may be an upright orientation, where the collar 320 (and thus the air process port(s) 332) may be above the container 310. A second orientation of the fluid device 300 may be an inverted orientation, where the collar 320 (and thus the air process port(s) 332) may be below the container 310. When the container 310 is carrying a liquid and the fluid device 300 is in the upright position, gravity may keep the liquid from contacting an interior of the collar 310. Oppositely, when the container 310 is carrying a liquid and the fluid device 300 is in the inverted position, gravity may cause the liquid to contact the interior of the collar 310 (e.g., a bottom interior surface and/or inner components thereof). Additionally, in some variations, the valve, such as a pinch valve, of the air process port 332 may need to be transitioned from an open configuration to a closed configuration (e.g., to isolate the in-line filter), which may take several seconds (e.g., about 0.5 to about 10 s, such as about 1 s to about 9 s, about 1.5 s to about 8 s, about 2 s to about 7 s, about 2.5 s to about 6 s, about 3 s to about 5 s, or about 3.5 s to about 4 s, including all ranges and subranges in-between). Thus, when the fluid device is in the inverted orientation and the valve of the air process port 332 requires transitioning from the open configuration to the closed configuration, there may be a risk of liquid from the container 310 leaking through the valve of the air process port 332. This leaking could clog the in-line filter (potentially preventing or minimizing the effects of the air “purge”), and could also reduce the accuracy of transfer operations and/or lead to cross-contamination between subsequent fluid transfers using the fluid device 300 due to residual fluid within the valve or filter. Accordingly, in some instances, the valved air process port 332 may include a ball valve configured to prevent fluid from traveling through the air process port 332 and to the filter during operations with the fluid device 300 in an inverted orientation. The ball valve may prevent fluid from flowing through a particular fluid pathway within the valve that leads to the filter, as will be explained in detail with reference to FIGS. 10-12B herein.

In some embodiments, the fluid conduits 322 of the collar 320 may fluidically couple each of the components of the collar 320, including the sterile liquid transfer port 324, the fluid access port 334, the fluid pump module 326, the fluid transport feature 338, the sterilization process ports 330, and the at least one air process port 332, and the container 310 in any suitable combination.

In some variations, the fluid conduits 322 may be tubing. In some variations, the fluid conduits 322 may be channels formed within a body of the collar 320. For example, the channels may be etched into, or be integrally formed with, a surface of the body of the collar 320, and a substrate may be coupled to the open face of the channels to provide a fluid seal. In some variations, the channels may be formed wholly within the body of the collar 320.

In some variations, the fluid transport feature 338 of the collar 320 comprises a venting tube, a liquid flow tube, and a fluid port. In some variations, the fluid transport feature 338, which is couplable to the opening 312 of the container 310, may be shaped and sized based on a shape and size of the opening 312 of the container 310. In some variations, the venting tube is configured to extend through the opening 312 of the container 310 and to be disposed within the container 310. The venting tube may extend from the opening 312 substantially into an open volume of the container 310 and may, via the fluid conduits 322 of the collar 320, provide an air connection between the container 310 and the external environment of the fluid device 300. In some variations, the venting tube further comprises a liquid vent reservoir configured to capture, upon inversion of the fluid device 300, fluid trapped within the venting tube. In some variations, a volume of the liquid vent reservoir is at least larger than a trappable volume of fluid within the venting tube. In this way, the venting tube and the liquid vent reservoir enable the fluid device 300 to be inverted without concern for fluid leakage and/or damage to any component of the fluid device 300 (e.g., air filters). In some embodiments, the liquid flow tube of the fluid transport feature 338 may be configured to extend through the opening 312 of the container 310 and to be disposed within the container 310. The liquid flow tube may extend from the opening 312 substantially into the open volume of the container 310. The liquid flow tube may provide a liquid connection between the container 310 and the external environment of the fluid device 300. For example, the liquid flow tube may be connected to the fluid access port 334 and may permit filling and/or depleting of liquid within the container 310. In some variations, the fluid port of the fluid transport feature 338 may comprise an aperture within a body of the fluid transport feature 338. The fluid port may fluidically connect the container 310 to the sterile liquid transfer port 324 and/or to the at least one air process port 332 via the fluid conduits 322. The fluid port may be used when the fluid device 300 is inverted to allow fluid transfer between the container 310 and another fluid device or cartridge via the sterile liquid transfer port 324. Similarly, when the fluid device 300 is upright, the fluid port may be used to flow fluid out of the container 310 and into e.g., a sample collection fluid device via the fluid access port 334 and the liquid flow tube.

In some variations, the collar 320 of the fluid device 300 further comprises a pressure relief valve proximate the outlet port of the compressible fluidic tubing of the fluid pump module 326. The outlet port may be in further fluid communication with the container 310 such that, when there is excessive pressure at the outlet port, fluid can be flowed into the container 310.

In some variations, the fluid device 300 can be reusable. For example, the fluid device 300 can be reusable when the fluid device 300 is used for automated transfer of fluids related to a single patient. For example, a single fluid device can be used to provide culture medium or other reagents to a cartridge comprising patient cells and, subsequently, can be used to retrieve samples of cellular solution from the cartridge for downstream analysis (such as quality control operations via e.g., the QC instrument of the workcell).

Turning now to FIG. 3B, a schematic diagram of an illustrative system for automated fluid transfer, including the fluid device 300 of FIG. 3A, is provided.

In some variations, a system 370 for automated fluid transfer comprises a workcell 350 and fluid device 300. Fluid device 300 comprises a container 310 and a collar 320, as described above. For brevity, additional, redundant description of the fluid device 300 will be provided only as necessary to aid in the description of the system 370. In some variations, the workcell 350 of the system 370 comprises a robot 340, a controller 360, a cam 327, a sterilant source 331, an air source 335, and sensor(s) 351. In some variations, the sensor(s) 351 may comprise or otherwise use imaging devices that utilize visible light, ultrasound, and fluorescence and/or devices configured to sense temperature, moisture, electricity, and the like. The robot 340 and the controller 360 may be substantially similar to those described above with reference to the workcell 110 and 203 of FIG. 1A through FIG. 2D. Additionally, or alternatively, the robot 340 and the controller 360 may be provided by a sterile liquid transfer instrument of the workcell 350.

In some variations, the fluid device 300 may be moved or otherwise manipulated by the robot 340 of the workcell 350 under the control of the controller 360. Instructions provided by the controller 360 may be influenced by data received from each of the collar 320, the cam 327, the robot 340, and the sensor(s) 351, or combinations thereof, and processed at the controller 360.

In some variations, data received by the controller 360 from the collar 320 of the fluid device 320 may include data corresponding to activity of the fluid pump module 326, the sterile liquid transfer port 324, the sterilization process ports 330, and/or the at least one air process port 332. Data received by the controller 360 from the cam 327 may include rotations per minute, a direction of rotation, fault detection data, and the like. Such data can be used in conjunction with known characteristics of the fluid conduits 322, such as a material, a length, and a diameter, and the compressible fluidic tubing of the fluid pump module 326 to determine or estimate a flow rate into and/or out of the container 310. This data may include properties (e.g., viscosity) of the fluid to be transferred. Data received by the controller 360 from the sensor(s) 351 may include optical data obtained via the at least one viewing window 336. The optical data may include absorbance, reflectance, and/or fluorescence of a fluid within the fluid conduits 322. Such data may be used by the controller 360 to determine transitions from liquid to air and from air to liquid. Data received by the controller 360 from the robot 340 may include positional data, identifying data relating to effected cartridges and/or fluid devices, and the like.

In some variations, wherein the fluid pump module 326 of the collar 320 comprises components of a peristaltic pump, the controller 360 may generate signals to the cam 327 to control fluid transfer between the fluid device 300 and another fluid device or cartridge by controllably compressing the compressible fluidic tubing of the fluid pump module 326. In some variations, a rotational velocity and a direction of rotation of the cam 327 can be controlled in order to control bidirectional flow and flow rate of a fluid. For example, a relationship between a cross sectional area of the fluid conduits 322 (which may be based on a diameter of the fluid conduits 322), a length of the fluid conduits 322, rheological properties of a fluid within the fluid conduits 322, and the rotational velocity of the cam 327 can be exploited as a function of time to control a flow rate into and/or out of the container 310.

In some variations, the controller 360 sends signals to the robot 340 to move and/or manipulate the fluid device 300 via the one or more robot engagement features 328 of the collar 320. Movement and manipulation of the device can include moving the fluid device 300 within the workcell 350, such as between a reagent vault of the workcell 350 and a sterile liquid transfer instrument of the workcell 350, and/or controlling an orientation of the fluid device 300. For example, the controller 360 may transmit a signal to the robot 340 to position the fluid device 300 in a particular orientation, such as in an upright orientation or an inverted orientation. In some variations, the fluid device 300 may be configured to be in the upright orientation when stored within a reagent vault (e.g., reagent vault 118 of FIG. 1A). In some variations, the fluid device 300 may be configured to be in the inverted orientation when coupled to an instrument (e.g., instrument 112 of FIG. 1A), such as a sterile liquid transfer instrument, to transfer fluid to a cartridge (e.g., cartridge 114 of FIG. 1B) that is interfacing with the instrument.

In some variations, the controller 360 generates and/or sends further signals to the robot 340 to manipulate the sterile liquid transfer port 324 of the collar 320 of the fluid device 300 to allow sterile, automated, and precisely metered (e.g., precise control of a transferred fluid volume) fluid transfer. For example, the signals to the robot 340 may control a configuration of the at least one port and valve of the sterile liquid transfer port 324. In some variations, and as it relates to opening a flow path between the sterile liquid transfer port 324 of the fluid device 300 and a sterile liquid transfer port of another fluid device, the controller 360 may first be configured to generate a port signal to the robot 340 to couple the at least one port to a corresponding port of the sterile liquid transfer port of the other fluid device. Coupling the at least one port to the corresponding port may comprise transitioning the ports to an open position. Next, the controller 360 may generate a valve signal to the robot 340 to translate the valve relative to a corresponding valve of the sterile liquid transfer device of the other fluid device. To finally open the fluid pathway, the controller 360 may then generate another valve signal to transition the valve and the corresponding valve to the open configuration. After fluid transfer, similar controlling signals can be generated to transition the sterile liquid transfer port 324 of the fluid device 300 to a closed configuration.

In some variations, the controller 360 may generate and/or send signals to the sterilization process ports 330 and/or the one or more air process ports 332 to control the flow of fluid therethrough. As noted above, the sterilization process ports 330 may be coupled to passive valves and may be configured to facilitate delivery of sterilant from the sterilant source 331 within the workcell 350 to the fluid conduits 322 and the sterile liquid transfer port 324 of the collar 320. In some variations, the workcell 350 may further comprise a fluid pump configured to control the flow of sterilant into and out of the fluid device 300 based on instructions received from the controller 360. For instance, after coupling of the fluid device 300 to another fluid device but before fluid transfer has begun, the sterile liquid transfer port 324 and fluid conduits 322 can be sterilized. This sterilization may include flowing the sterilant, under the guidance of the controller 360 via the fluid pump, from the sterilant source 331, into and out of the sterile liquid transfer port 324 via the fluid conduits 322.

In some variations, the at least one air process port 332 is valved (e.g., pinch valves) and may provide a pathway for air to enter or leave the fluid device 300 during filling of the container 310 and/or depleting of the container 310. In some variations, the at least one air process port 332 may be connected to an air source 335 of the workcell 350. In some variations, the air source 335 may comprise compressed air which can be used to purge the fluid conduits 322 of the collar 320 before and/or after fluid transfer therethrough. In some variations, the workcell 350 may further comprise a fluid pump configured to control the flow of air from the air source 335 into and out of the fluid device 300 via the at least one air process port 332 based on instructions received from the controller 360. For example, after fluid transfer between the fluid device 300 and another fluid device via the fluid pump module 326 of the collar 320, compressed air can be delivered within the fluid conduits 322 to ensure complete transfer of the fluid. In some variations, this air “purge” can be performed by delivering air to the fluid conduits 322 via the at least one air process port 332. In other variations, this air “purge” can be performed by delivering, via the at least one air process port 332, air through the fluid transport feature 338 and into the container 310. The compressed air may be delivered through the air process port 332 at a force of about 0.25 psi to about 50 psi, such as about 0.5 psi to about 40 psi, about 0.75 psi to about 30 psi, about 1 psi to about 20 psi, about 2 psi to about 18 psi, about 3 psi to about 16 psi, about 4 psi to about 14 psi, about 5 psi to about 12 psi, about 6 psi to about 10 psi, about 7 psi to about 9 psi, or at about 8 psi (including all ranges and subranges therebetween). For example, the air “purge” may be performed at greater than or equal to about 8 psi to unclog a filter coupled to the air process port 332. In some variations, the air “purge” may have a duration of about 1 s to about 5 min, such as about 1.5 s to about 4 min, about 2 s to about 3 min, about 3 s to about 2 min, about 4 s to about 1 min, about 5 s to about 45 s, about 6 s to about 30 s, about 8 s to about 20 s, or about 10 s to about 15 s (including all ranges and subranges therebetween).

In some variations, data from each of the components of the workcell 350 described above can be integrated to perform automated fluid transfer. For example, the controller 360 may generate and/or send a signal to the robot 340 to move, invert, and couple the fluid device 300 to another fluid device or cartridge via the one or more robot engagement features 328. Coupling the fluid device 300 to another fluid device or cartridge includes apposition of respective sterile liquid transfer ports. Each of the respective sterile liquid transfer ports may comprise mechanical seals that are in sealing contact when the sterile liquid transfer ports are connected. These mechanical seals help ensure a first mechanism for sterility. The controller 360 may generate and/or send a signal to the robot 340 to actuated corresponding ports of the respective sterile liquid transfer ports to an open position. The controller 360 may then send a signal to a fluid pump associated with the sterilant source 331 to circulate sterilant within the collar 320 and, thus, within the sterile liquid transfer port 324 and the interface between the respective sterile liquid transfer ports. This may help ensure a second mechanism for sterility. After removal of the sterilant, the controller 360 may generate and/or send a signal to the robot 340 to manipulate corresponding valves of the respective sterile liquid transfer ports to open a fluid pathway between the fluid device 300 and the other fluid device. With the fluid pathway open, the controller 360 may generate and/or send a signal to the cam 327 to begin rotating in a particular direction. When the controller 360 receives data from the sensor(s) 351 indicating an air to liquid transition has been detected via the at least one viewing window 336, the controller 360 may momentarily halt the cam 327 in order to determine a particular direction, a particular velocity, and a particular duration at which the cam 327 should be rotated in order to generate a controlled fluid flow rate within the fluid conduits 322 of the collar 320 that achieves a desired transferred fluid volume. After the particular duration has passed, or when the controller 360 receives data from the sensor(s) 351 indicating that a liquid to air transition has been detected via the at least one window 336, the controller 360 may generate a signal to halt the cam 327. The controller 360 may then generate and/or send a signal to a fluid pump associated with the air source 335 to supply air to the fluid conduits 322 to purge the line and ensure completion of the fluid transfer. After this air “purge”, the controller 360 may generate decoupling signals so that the robot 340 disconnects the sterile liquid transfer port 324 of the fluid device 300 from a corresponding sterile liquid transfer port of another fluid device and re-orients (e.g., repositions the fluid device 300 to an upright orientation) and moves the fluid device 300 to another location.

Turning now to FIG. 4A through FIG. 4I, renderings of views of an illustrative fluid device for automated fluid transfer are provided. Like reference numerals will be used with reference to FIG. 4A through FIG. 4I, though, not all features will be called out in each figure.

In some embodiments, the fluid device 400 comprises a container 410 and a collar 420. As shown in FIG. 4F, the container 410 may comprise an opening 412 and at least one collar coupling feature 403. In some variations, the opening 412 of the container 410 comprises an annular seal. As shown in FIG. 4A through FIG. 4D, the collar 420 may comprise one or more robot engagement features such as protrusion 429, as well as fluid conduits 422, a sterile liquid transfer port 424 having a mechanical seal 425, at least one container coupling feature 402 couplable to a corresponding one of the at least one collar coupling feature 403 of the container 410, at least one viewing window 436, a fluid pump module 426 having compressible fluidic tubing 467, a plurality of ports including sterilization process port(s) 430 (“9”, “10”) and at least one air process port 432 (“5”), and a fluid access port 434.

In some variations, the compressible fluidic tubing 457 may be coupled between an outlet port 415 of the collar 420 which delivers fluid to the compressible fluidic tubing 457 and an inlet port 414 of the collar 420 which returns the fluid to the fluid conduits 422 of the collar 420. As can be seen in FIG. 4A, the compressible fluidic tubing 457 may be proximate an external surface of the collar 420 so that an affector of the workcell may interact with the compressible fluidic tubing 457 to move fluid therein. For example, the external surface of the collar 420 may be curved proximate the compressible fluidic tubing 457 so that a cam of the workcell may compress the compressible fluidic tubing 457.

In some variations, and as shown in FIG. 4B, the at least one viewing window 436 is an aperture providing a vantage to at least a segment of the fluid conduits 422 of the collar 420 and permitting optical evaluation of fluid movement within the fluid conduits 422. For example, a sensor disposed on the sterile liquid transfer instrument of the workcell may be aligned with the at least one viewing window 436 of the collar 420 and may detect a transition from air to liquid within the fluid conduits 422, thereby indicating the beginning of metered fluid transfer, or may detect a transition from liquid to air within the fluid conduits 422, thereby indicating an emptying of the container 410. In some variations, the sensor may be a bubble sensor 1 and a bubble sensor 2 disposed within the collar 420. The bubble sensors 1, 2 may be configured to detect a transition from air to liquid within the fluid conduits 422, thereby indicating the beginning of metered fluid transfer, or may be configured to detect a transition from liquid to air within the fluid conduits 422, thereby indicating an emptying of the container 410.

In some variations, the sterile liquid transfer port 424 of the collar 420 may comprise at least one of a port 417 and a valve (not shown) and may form one part of a sterile fluid pathway between the fluid device 400 and another fluid device and/or cartridge to enable sterile, automated, and precisely metered (e.g., precise control of a transferred fluid volume) fluid transfer. As shown in FIG. 4A, FIG. 4B, and FIG. 4C, the sterile liquid transfer port 424 may comprise a mechanical seal 425. The mechanical seal 425 helps provide sterility of a fluid transfer pathway between the fluid device 400 and the other fluid device or cartridge. In some variations, a robot of the workcell, which may be a robot of a sterile liquid transfer instrument, may be configured to manipulate the fluid device 400 via the protrusion(s) 429 and alignment features 418 of the sterile liquid transfer port 424 to couple the fluid device 400 to the other fluid device or cartridge. Further, the robot may be configured to operate the sterile liquid transfer port 424 to open and close a set of ports and valves thereof, including the at least one of the port 417, to permit fluid flow between the fluid device 400 and a cartridge or another fluid device.

In some variations, and as shown in FIG. 4A, FIG. 4B, and FIG. 4C, the sterilization process ports 430 may be configured to deliver sterilant (e.g., vaporized hydrogen peroxide (“VHP”)) from a sterilant source within the workcell to the fluid conduits 422 and the sterile liquid transfer port 424 (e.g., via a sterilant input 8) of the collar 420. In some variations, the at least one air process port 432 may provide a pathway for air to enter or leave the fluid device 400 during filling of the container 410 and/or depleting of the container 410. In some variations, the at least one air process port 432 may be connected to an air source (e.g., atmospheric air). In some variations, the air source may comprise compressed air, which can be used to purge the fluid conduits 422 of the collar 420 (via air purge tube 4) before and/or after fluid transfer therethrough.

In some variations, the at least one air process port 432 may comprise in-line filters. Accordingly, in some embodiments, the fluidic device can advantageously include at least two means of sterilization such as, for example, via the mechanical seal 425 and via the delivery of at least one sterilant.

In some variations, the fluid conduits 422 of the collar 420, which may include transfer tube 6 (fluid input to the fluid pump module 426), transfer extension 18 (fluid from the container 510), transfer end 3 (fluid into the sterile liquid transfer port 424), sterilization input 8 (leading into the sterile liquid transfer port 424), and sterilization output 7 (leading out of the sterile liquid transfer port 424), may fluidically couple each of the components of the collar 420, including the sterile liquid transfer port 424, the fluid access port 434, the fluid pump module 426, the fluid transport feature 438, the sterilization process ports 430, and the at least one air process port 432, and the container 410 in any suitable combination. As in FIG. 4A through FIG. 4D, the fluid conduits 422 may be tubing.

In some variations, the fluid conduits 422 of the collar 420 may be fluidically coupled to a container 410 via a fluid transport feature 438 couplable to an opening 412 of the container 410, as shown in FIG. 4E. In particular, FIG. 4E shows the fluid transport feature 438 (“13/14”) connected to the opening 412 of the container 410 but disconnected from the remaining fluid conduits 422 of the collar 420. The fluid transport feature 438 comprises a venting tube 442 (“15”), a liquid flow tube 443 (“17”), and a fluid port 446 (“16”). In some embodiments, the fluid transport feature 438, which is couplable to the opening 412 of the container 410, may be shaped and sized based on a shape and size of the opening 412 of the container 410. For example, the fluid transport feature 438 may be substantially circular and/or cylindrical. In some variations, the venting tube 442 is configured to extend through the opening 412 of the container 410 and to be disposed within the container 410. The venting tube 442 may extend from the opening 412 substantially into an open volume of the container 410 and may, via the fluid conduits 422 of the collar 420, provide an air connection between the container 410 and the external environment of the fluid device 400.

In some variations, the venting tube 442 further comprises a liquid vent reservoir configured to capture, upon inversion of the fluid device 400, fluid trapped within the venting tube 442. In some variations, a volume of the liquid vent reservoir is at least larger than a maximum trappable volume of fluid within the venting tube 442. In this way, the venting tube 442 and the liquid vent reservoir enable the fluid device 400 to be inverted without concern for fluid leakage and/or damage to any component of the fluid device 400 (e.g., saturation of air filters). In some embodiments, the liquid flow tube 443 of the fluid transport feature 438 may be configured to extend through the opening 412 of the container 410 and to be disposed within the container 410. The liquid flow tube 443 may extend from the opening 412 substantially into the open volume of the container 410. The liquid flow tube 443 may provide a liquid connection between the container 410 and the external environment of the fluid device 400. For example, the liquid flow tube 443 may be connected to the fluid access port 434 and may permit filling and/or depleting of liquid within the container 410. In some variations, the fluid port 446 of the fluid transport feature 438 may comprise an aperture within a body of the fluid transport feature 438. The fluid port 446 may fluidically connect the container 410 to the sterile liquid transfer port 424 and/or to the at least one air process port 432 via the fluid conduits 422. In one variation, the fluid port 446 may be used when the fluid device 400 is inverted to allow fluid transfer out of the container 410 and into another fluid device or cartridge via the sterile liquid transfer port 424. In another variation, when the fluid device 400 is upright, the fluid port 446 may be used to flow fluid out of the container 410 and into e.g., a sample collection fluid device via the fluid access port 434 and the liquid flow tube 443.

In some variations, the collar 420 of the fluid device 400 further comprises a pressure relief valve (not shown) proximate the outlet port 415 of the compressible fluidic tubing 457 of the fluid pump module 426. The outlet port 415 may be in further fluid communication with the container 410 such that, when there is excessive pressure at the outlet port 415, fluid can be flowed into the container 410.

In some variations, the container 410 of the fluid device 400 further comprises a user grasping feature(s) 454 to permit the user to manually manipulate the container 410 and/or fluid device 400, as needed. For example, the user grasping feature(s) 454 may enable the user to make a snap fit between the container 410 and the collar 420.

With reference now to FIG. 4G through FIG. 4I, illustrative renderings of engagement between a robot of a workcell and an illustrative fluid device for automated fluid transfer are provided. In particular, FIG. 4G through FIG. 4I provide an exemplary grasping mechanism by which a fluid device may be manipulated by a robot of a workcell. In some variations, the robot of the workcell may be a robot of a sterile liquid transfer instrument. However, for clarity, the grasping described below will be performed generally within a workcell, wherein a controller (such as controller 360 of FIG. 3B) controls grasping.

As shown in FIG. 4G, engagement, or grasping, between a robot 440 of a workcell and a fluid device 400 includes coupling between a robot grasping feature 453 of the robot 440 and at least one of the robot engagement features 428 of the fluid device 400. In some variations, the coupling between the robot 440 and the fluid device 400 comprises receiving the at least one robot engagement feature 428 within an aperture of the robot grasping feature 453. As partially shown in FIG. 4H, the at least one robot engagement feature 428 may be received within an opening 473 of the robot grasping feature 453. FIG. 4I is a cross-sectional view of an illustrative rendering of the robot grasping feature 453, showing a first clamp 474 and a second clamp 475 configured to be translated relative to the opening 473. Specifically, after the at the least one robot engagement feature 428 of the fluid device 400 is received within the opening 473 of the robot grasping feature 453, the first clamp 474 and the second clamp 475 may be translated toward the opening 473 to grasp the at least one robot engagement feature 428 and, therefore, the fluid device 400. After the clamps 474, 475 are engaged with the fluid device 400 via the at least one robot engagement feature 428, the robot 440 may manipulate (e.g., rotate, translate, invert) the fluid device 400 as required by the methods described herein.

Turning now to FIG. 5A through FIG. 5L, renderings of views of another illustrative fluid device for automated fluid transfer are provided. Like reference numerals will be used with reference to FIG. 5A through FIG. 5L, though, not all features will be called out in each figure.

In some variations, the fluid device 500 comprises a container 510 and a collar 520. As shown in FIG. 5I, the container 510 may comprise an opening 512 and at least one collar coupling feature 503. In some variations, the opening 512 of the container 510 comprises an annular seal. As shown in FIG. 5A through FIG. 5E, the collar 420 may comprise one or more robot engagement features such as depression(s) 529, as well as fluid conduits 522, a sterile liquid transfer port 524 having a mechanical seal 525, a fluid transport feature 538 couplable to the opening 512 of the container 510, at least one container coupling feature 502 couplable to a corresponding one of the at least one collar coupling feature 503 of the container 510, at least one viewing window 536, a fluid pump module 526 having compressible fluidic tubing, a plurality of ports including sterilization process port(s) 530 and at least one air process port 532, and a fluid access port 534.

In some variations, the compressible fluidic tubing (not shown) may be coupled between an outlet port 515 of the collar 520 which delivers fluid to the compressible fluidic tubing and an inlet port 514 of the collar 520 which returns the fluid to the fluid conduits 522 of the collar 520.

In some variations, and as shown in FIG. 5B, the at least one viewing window 536 can be an aperture providing a vantage to at least a segment of the fluid conduits 522 of the collar 520 and permitting optical evaluation of fluid movement within the fluid conduits 522. For example, a sensor disposed on the sterile liquid transfer instrument of the workcell may be aligned with the at least one viewing window 536 of the collar 520 and may detect a transition from air to liquid within the fluid conduits 522, thereby indicating the beginning of metered fluid transfer, or may detect a transition from liquid to air within the fluid conduits 522, thereby indicating an emptying of the container 510.

In some variations, the sterile liquid transfer port 524 of the collar 520 may comprise at least one of a port 517 and a valve (not shown) and may form one part of a sterile fluid pathway between the fluid device 500 and another fluid device and/or cartridge to enable sterile, fully automated, and precisely metered (e.g., precise control of a transferred fluid volume) fluid transfer. As shown in FIG. 5A, FIG. 5B, and FIG. 5C, the sterile liquid transfer port 524 may comprise a mechanical seal 525. The mechanical seal 525 may help provide sterility of a fluid transfer pathway between the fluid device 500 and another fluid device or cartridge. In some variations, a robot of the workcell, which may be a robot of a sterile liquid transfer instrument, may be configured to manipulate the fluid device 500 via the depression(s) 529 and alignment features 518 of the sterile liquid transfer port 525 to couple the fluid device 500 to the other fluid device or cartridge. Further, the robot may be configured to operate the sterile liquid transfer port 524 to open and close a set of ports and valves thereof, including the at least one of the port 517, to permit fluid flow between the fluid device 500 and the cartridge or the other fluid device.

In some variations, and as shown in FIG. 5A, FIG. 5B, and FIG. 5C, the sterilization process ports 530 may be configured to deliver sterilant (e.g., vaporized hydrogen peroxide (“VHP”)) from a sterilant source within the workcell to the fluid conduits 522 and the sterile liquid transfer port 524 of the collar 520. In some variations, the at least one air process port 532 may provide a pathway for air to enter or leave the fluid device 500 during filling of the container 510 and/or depleting of the container 510. In some variations, the at least one air process port 532 may be connected to an air source (e.g., atmospheric air). In some variations, the air source may comprise compressed air, which can be used to purge the fluid conduits 522 of the collar 520 before and/or after fluid transfer therethrough. In some variations, the at least one air process port 532 may comprise in-line filters, such as filter(s) 533. The filter(s) 533 may be hydrophobic filters.

The valved air process ports herein may risk leaking fluid (e.g., liquid) from the container when the fluid device is in certain orientations, such as during operations with the fluid device in an inverted orientation, as explained above with reference to FIG. 3B. Accordingly, in some instances, the valved air process ports herein may include a ball valve configured to prevent liquid from traveling through the port and to the filter when the fluid device is in an inverted orientation. That is, the ball valve may prevent fluid from flowing through a particular fluid pathway within the valve that leads to the filter. In some variations, the fluid device may be configured to be in the inverted (e.g., container-side-up) orientation when coupled to an instrument of the workcell (e.g., a sterile liquid transfer instrument) for a fluid transfer operation involving a cartridge that is interfacing with the instrument. In some variations, the ball valve may be mounted on a collar of the fluid device. Any suitable mechanism may be used to attach the ball valve to the collar, such as welding (e.g., laser welding), adhesive(s), fastener(s), and/or the like. In some variations, the ball valve and the collar may be integrated (e.g., of a single construction). An exemplary such ball valve 1002 is shown in FIG. 10. As depicted, the ball valve 1002 may be coupled to a manifold 1004 of a fluid device collar 1000 (e.g., on a bottom interior surface thereof).

In particular, the ball valves herein may include a housing having an inlet, a hollow interior, and an outlet (creating a path for fluid flow toward the filter), and may be configured to carry a ball therein. In some variations, the housing may additionally include a top cover. The housing may be made of any suitable material, such as metal (e.g., aluminum) and/or plastic. In some variations, the housing may be constructed of metal (e.g., aluminum), and may be machined (with or without the collar of the fluid device). Additionally, the housing may have any suitable geometry. For example, the housing may have one or more straight sidewalls (e.g., 1, 2, 3, 4, 5 or more than 5 straight sidewalls, such as 4 straight sidewalls) and/or one or more rounded sidewalls (e.g., 1, 2, 3, 4, 5 or more than 5 straight sidewalls, such as 3 rounded sidewalls). The hollow interior of the housing may have a constant or varied width, such as a width of about 1 mm to about 5 mm, such as about 1.5 mm to about 4.75 mm, about 2 mm to about 4.5 mm, about 2.5 mm to about 4.25 mm, or about 3 mm to about 4 mm (including all ranges and subranges therebetween). Moreover, a height of the housing may be constant or varied, and may be about 1 mm to about 20 mm, such as about 1.5 mm to about 15 mm, about 2 mm to about 10 mm, about 2.5 mm to about 9 mm, or about 3 mm to about 8 mm, about 3.5 mm to about 7 mm, about 4 mm to about 6 mm, or about 4.5 mm to about 5 mm (including all ranges and subranges therebetween). In some variations, one or both of the inlet and outlet of the housing may extend from a surface thereof, such as from a bottom surface of the housing. For example, one or both of the inlet and outlet may have an exterior extending from the housing and a lumen therethrough. The exterior of the inlet and/or outlet may be any suitable shape, such as cylindrical or rectangular. In some variations, the exterior of the inlet and/or outlet may have a constant or varied height of 1 mm to about 10 mm, such as about 1.5 mm to about 9 mm, about 2 mm to about 8 mm, about 2.5 mm to about 7 mm, or about 3 mm to about 6 mm, about 3.5 mm to about 5 mm, or about 4 mm to about 4.5 mm (including all ranges and subranges therebetween). Moreover, the lumen of the inlet and/or outlet may have a constant or varied width or diameter of between about 1 mm to about 5 mm, such as about 1.5 mm to about 4.75 mm, about 2 mm to about 4.5 mm, about 2.5 mm to about 4.25 mm, or about 3 mm to about 4 mm (including all ranges and subranges therebetween). In some variations, a portion (e.g., at least a portion) of the lumen of one or both of the inlet and the outlet may be tapered. As an example, the outlet may have a lumen with a first portion having a first width or diameter, a second portion having a second, smaller width or diameter, and a tapered portions. The first width or diameter may be between about 3 mm and about 4 mm, such as about 3.7 mm, and the second width or diameter may be between about 1 mm and about 2 mm, such as about 1.6 mm. As another example, the outlet may have a lumen with the first portion having the first width or diameter, and the second portion having the smaller width or diameter may extend directly from the first portion.

Moreover, the housing of the ball valve may be configured to carry a ball within the outlet of the housing, such as within a first portion (e.g., having a largest width or diameter) of a lumen of the outlet. In some variations, a diameter of the ball may be about equal to or less than a first width or diameter of the first portion of lumen of the outlet, and may be about equal to or greater than a second width or diameter of a second (e.g., lower) portion of the lumen. Accordingly, the ball may be configured to settle between the first and second portions of the lumen of the outlet when the fluid device is in the inverted orientation, thereby partially (e.g., at least partially) blocking fluid flow through the lumen of the outlet (and thus blocking fluid from leaking onto the in-line filter). Further, the ball may be made of any suitable material, such as metal, plastic, or rubber (e.g., silicone).

In some variations, the housing may further include a ball-stop to maintain a position of the ball within the outlet lumen when the fluid device is in an upright (e.g., container-side-down) orientation. The ball-stop may be shaped to allow fluid flow through the outlet lumen when the fluid device is in the upright and inverted orientations while preventing the ball from moving within the hollow interior of the housing. In particular, the ball-stop may include one or more conduits, such as a plurality of conduits, extending therethrough to allow airflow around the ball when the fluid device is in the inverted orientation, thus allowing for an air “purge” procedure during operations when the fluid device is inverted. A plurality of conduits of the ball-stop may include 2, 3, 4, 5, or more than 5 fluid conduits. In some variations, the ball-stop may have a plurality of legs, such as 2, 3, 4, 5, or more than 5 legs, and one or more of the legs (e.g., each leg) may include a conduit extending therethrough. The legs may have a length that is about equal to or less than a radius, or half a width of, the outlet lumen. In some variations, the ball-stop may be configured to be press-fit into the outlet lumen.

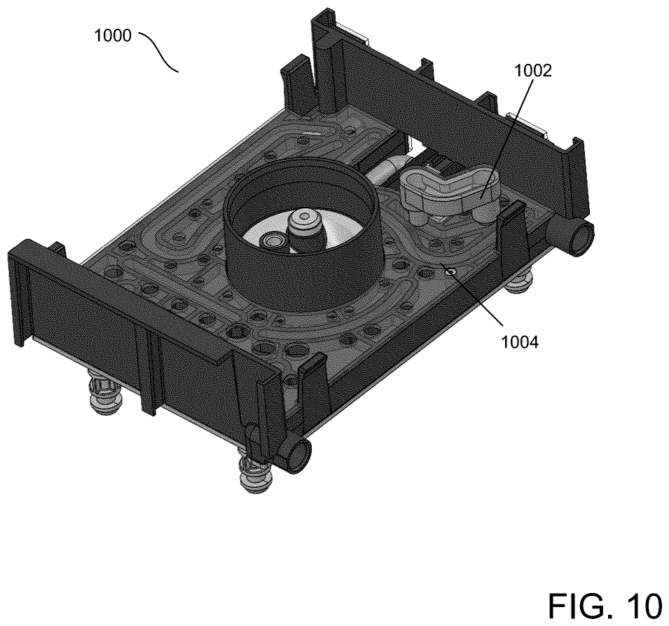

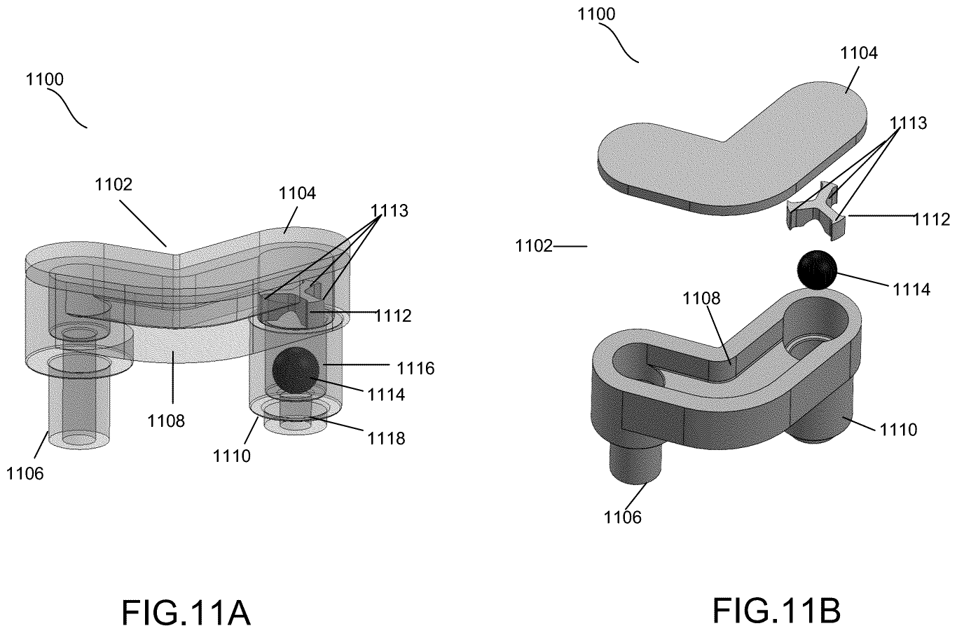

FIGS. 11A and 11B show variations of a ball valve 1100 in more detail. In FIG. 11A, a transparent view of the ball valve 1100 is depicted in an inverted orientation. In FIG. 11B, an exploded view of the ball valve 1100 is depicted in the inverted orientation. As shown in both FIGS. 11A and 11B, the housing 1102 of the ball valve 1100 includes top cover 1104, inlet 1106, hollow interior 1108, outlet 1110, ball-stop 1112, and ball 1114. The ball-stop 1112 includes a plurality of conduits 1213, such as 3 conduits extending through the ball-stop 1112. As shown in FIG. 11A, in the inverted orientation, the ball 1114 rests between a first portion 1116 of the outlet 1110 and a second portion 1118 of the outlet 1110 because the first portion 1116 has a lumen diameter that is greater than a lumen diameter of the second portion 1118.

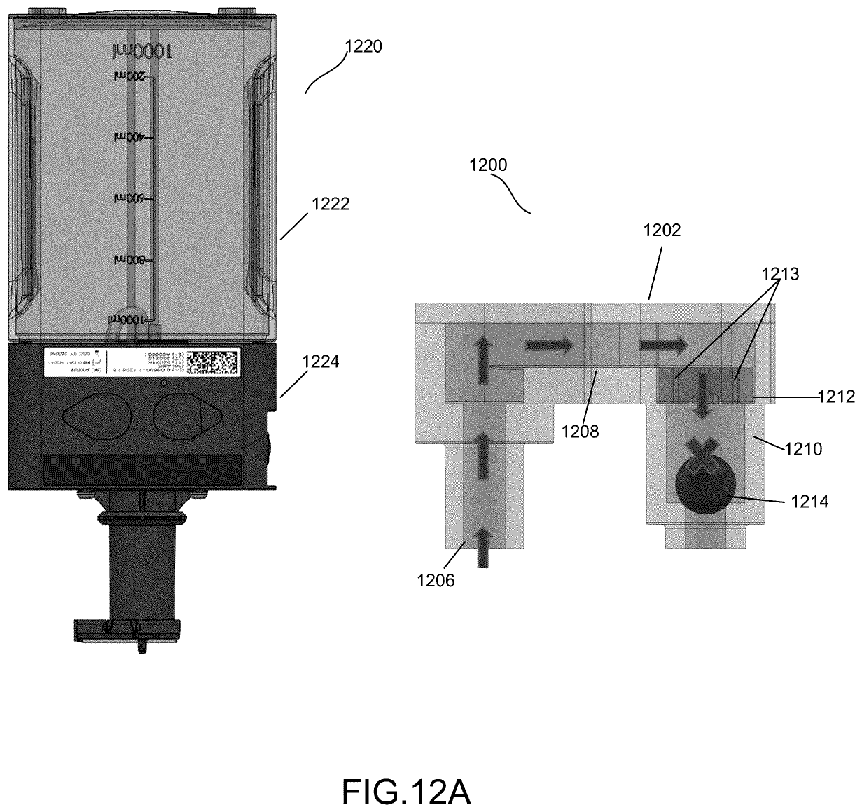

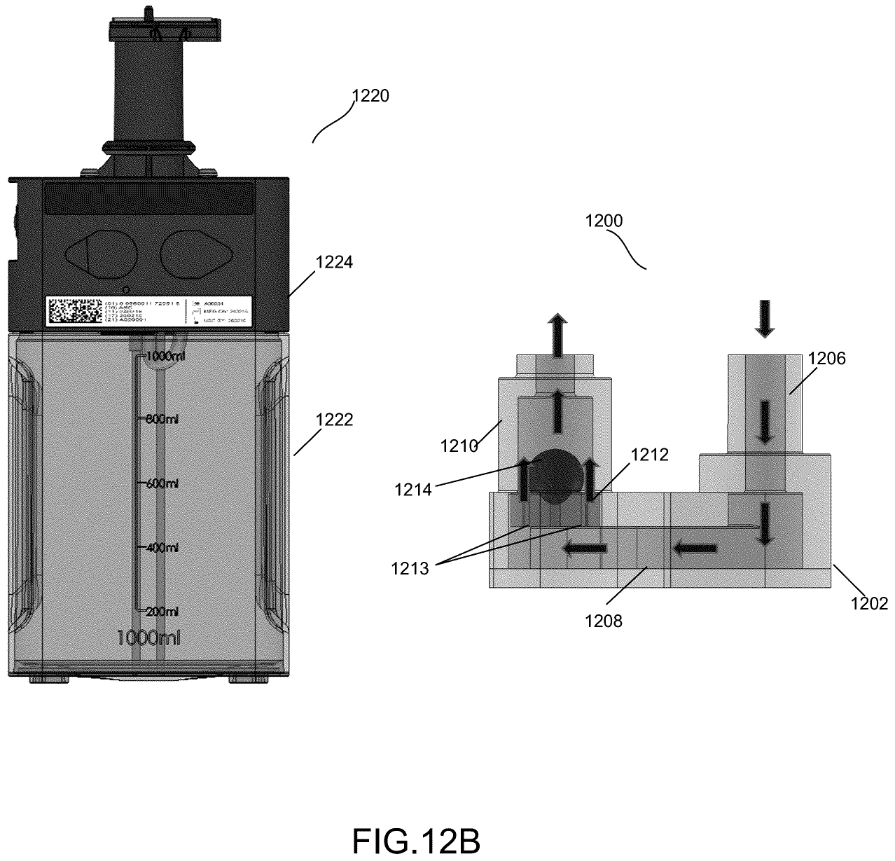

Similarly, FIG. 12A illustrates a fluid flow path through a ball valve 1200 when a fluid device 1220 having the ball valve 1200 is in an inverted position. As shown, in the inverted position, a container 1222 of the fluid device 1220 is above a collar 1224 of the fluid device 1220. In this position, fluid (e.g., liquid) may flow through the inlet 1206, hollow interior 1208 and ball-stop 1212 (e.g., via the conduits 1213 thereof) of the housing 1202. However, the ball 1214 stops the fluid from exiting the outlet 1210, thereby preventing the fluid from leaking onto a filter or other component coupled to the outlet 1210. Oppositely, FIG. 12B illustrates a fluid flow path through the ball valve 1200 when the fluid device 1220 an upright position. As shown, in the upright position, the container 1222 of the fluid device 1220 is below the collar 1224 of the fluid device 1220. In this position, fluid (e.g., liquid) may flow through the inlet 1206, hollow interior 1208 and ball-stop 1212 (e.g., via the conduits 1213 thereof) of the housing 1202. Additionally, because the ball 1210 rests on the ball-stop 1212 in this position, the fluid (e.g., air) may exit the outlet 1210.

Turning back to FIGS. 5A-5L, in some variations, the fluid conduits 522 of the collar 520 may fluidically couple each of the components of the collar 520, including the sterile liquid transfer port 524, the fluid access port 534, the fluid pump module 526, the fluid transport feature 538, the sterilization process ports 530, and the at least one air process port 532, and the container 510 in any suitable combination. As shown in FIG. 5D, which provides a cross sectional view of a body of the collar 520, and FIG. 5E, which shows a plan view of a bottom of the collar 520, the fluid conduits 522 may be channels 563 formed within the body of the collar 520. For instance, the channels 563 may be etched into a surface of the body of the collar 520, and a substrate 562 may be coupled to the open face of the channels 563 to provide a fluid seal. The channels 563 may fluidically couple each of the components of the collar 520 within the collar 520 and with the container 510, in any combination.

For instance, as shown in FIG. 5D and FIG. 5E, the fluid conduits 522 may form a three-dimensional network of channels 563 that enable movement of fluid within, into, and out of the collar 520 while maintaining a workable form factor and simplicity of user interactions. The channels 563 include a sterilization process channel 566 in connection with the at least one sterilization process ports 530 and the sterile liquid transfer port 524, an air venting channel 564 in connection with an air venting tube (e.g., venting tube 542), an air process channel 565 in connection with the at least one air process port 532, a volume of the container 510, and the sterile liquid transfer port 524, a pressure relief channel 567 in connection with e.g., the fluid pump module 526 and the volume of the container 510, a user fluid channel 568 in connection with the fluid access port 534 and the volume of the container 510, and a transport fluid channel 569 in connection with the volume of the container 510 and the sterile liquid transfer port 524, the transport fluid channel 569 being configured to enable the automated fluid transfer between the fluid device and another fluid device.

In some variations, the fluid transport feature 538 of the collar 520 comprises a venting tube 542, a liquid flow tube 546, a fluid port 543, a pressure relief port 548, and an annular seal 539. In some embodiments, the fluid transport feature 538, which is couplable to the opening 512 of the container 510 via a threadable coupling, may be shaped and sized based on a shape and size of the opening 512 of the container 510. For example, the fluid transport feature 538 may be substantially circular and/or cylindrical. Moreover, in addition to the annular seal of the opening 512 of the container 510, the annular seal 539 of the fluid transport feature 538 can minimize if not eliminate fluid leakage and potential contamination resulting therefrom.

In some variations, the venting tube 542 is configured to extend through the opening 512 of the container 510 and to be disposed within the container 510. The venting tube 542 may extend from the opening 512 substantially into an open volume of the container 510 and may, via the fluid conduits 522 of the collar 520, provide an air connection between the container 510 and the external environment of the fluid device 500. In some variations, the venting tube 542 further comprises a venting tube reservoir 544 configured to capture, upon inversion of the fluid device 500, fluid trapped within the venting tube 542. In some variations, a volume of the venting tube reservoir 544 is at least larger than a maximum trappable volume of fluid within the venting tube 542. In this way, the venting tube 542 and the venting tube reservoir 544 enable the fluid device 500 to be inverted without concern for fluid leakage and/or damage to any component of the fluid device 500 (e.g., air filters). Additional renderings of the venting tube 542 and aspects thereof are shown in FIG. 5G and FIG. 5H. In some embodiments, the venting tube 542 comprises the venting tube reservoir 544, a venting tube deflector 545, and a tube extending from the venting tube reservoir 544. Upon inversion of the fluid device 500, a trapped volume of fluid within the venting tube 542 flows toward the venting tube deflector 545 while air (indicated by green arrows) flows in an opposite direction. As shown in FIG. 5H, the trapped volume of fluid flowing toward the venting tube deflector 545 contacts the venting tube deflector 545 and is deflected to the side, away from the apertures of the venting tube deflector 545, and into a captive space of the venting tube reservoir 544. In this way, the venting tube 542 of the fluid transport feature 538 of the collar 520 of the present disclosure prevents the blockage of the venting tube 542 and/or blocking of an air filter otherwise coupled to the fluid conduits 522 and, thus, allows air to backfill the container 510 during fluid transfer.

In some variations and returning to FIG. 5A, FIG. 5B, and FIG. 5F, the liquid flow tube 546 of the fluid transport feature 538 may be configured to extend through the opening 512 of the container 510 and to be disposed within the container 510. The liquid flow tube 546 may extend from the opening 512 substantially into the open volume of the container 510. The liquid flow tube 546 may provide a liquid connection between the container 510 and the external environment of the fluid device 500. For example, the liquid flow tube 546 may be connected to the fluid access port 534 and may permit filling and/or depleting of liquid within the container 510.

In some variations, the fluid port 543 of the fluid transport feature 538 may comprise an aperture within a body of the fluid transport feature 538. The fluid port 543 may fluidically connect the container 510 to the sterile liquid transfer port 524 and/or to the at least one air process port 532 via the fluid conduits 522. In one variation, the fluid port 543 may be used when the fluid device 500 is inverted to allow fluid transfer out of the container 510 and into another fluid device or cartridge via the sterile liquid transfer port 524. In another variation, when the fluid device 500 is upright, the fluid port 543 may be used to flow fluid out of the container 510 and into e.g., a sample collection fluid device via the fluid access port 534 and the liquid flow tube 546.

In some variations, the collar 520 of the fluid device 500 further comprises a pressure relief valve proximate the outlet port 515 of the compressible fluidic tubing of the fluid pump module 526. The outlet port 515 may be in further fluid communication with the container 510 via the pressure relief valve 548 such that, when there is excessive pressure at the outlet port 515, fluid can be flowed into the container 510.

In some variations, the container 510 of the fluid device 500 further comprises a user grasping feature(s) 554 to permit the user to manually manipulate the container 510 and/or fluid device 500, as needed. For example, the user grasping feature(s) 554 may enable the user to make a snap fit between the container 510 and the collar 520.

With reference now to FIG. 5J through FIG. 5L, illustrative renderings of engagement between a robot of a workcell and an illustrative fluid device for automated fluid transfer are provided. In particular, FIG. 5J through FIG. 5L provide an exemplary grasping mechanism by which a fluid device may be manipulated by a robot of a workcell. In some variations, the robot of the workcell may be a robot of a sterile liquid transfer instrument. However, for clarity, the grasping described below will be performed generally within a workcell, wherein a controller (such as controller 360 of FIG. 3B) controls grasping.

As shown in FIG. 5J, engagement, or grasping, between a robot 540 of a workcell and a fluid device 500 includes coupling between a robot grasping feature 553 of the robot 540 and at least one of the robot engagement features (indicated by reference numeral 529 in FIG. 5A) of the fluid device 500, the robot engagement features being depressions, openings, and the like. In some variations, the coupling between the robot 540 and the fluid device 500 comprises sliding contact between a first clamp 574 and a second clamp 575 of the robot grasping feature 553 within respective ones of the robot engagement features, as better shown in FIG. 5K and FIG. 5L. In particular, as shown in FIG. 5K, a first coupling position of the clamps 574, 575 and respective robot engagement features 528 is shown. In the first coupling position, the first clamp 574 and the second clamp 575 are passed into an opening defined by the respective robot engagement features 528. After being inserted into the respective openings, the first clamp 574 and the second clamp 575 may be translated in opposite directions toward a second coupling position, thereby engaging the clamps 574, 575 with surfaces of the respective robot engagement features 528 and grasping the fluid device 500. After the clamps 574, 575 are engaged with the fluid device 500 via the at least one robot engagement feature 528, as shown in FIG. 5L, the robot 540 may manipulate (e.g., rotate, translate, invert) the fluid device 500 as required by the methods described herein.

### II. Automated Fluid Transfer Methods

Described herein are also methods for fluid transfer, for example, automated fluid transfer within a cell processing system.

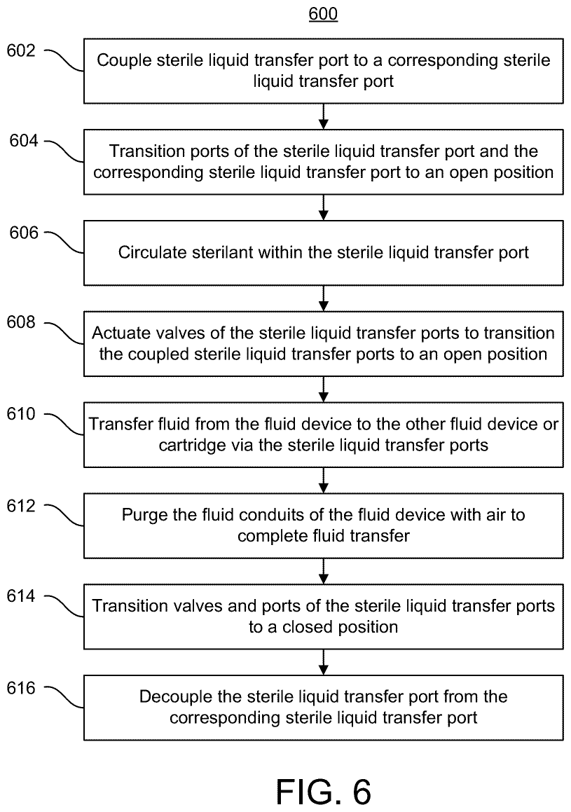

Initially, a framework for automated fluid transfer will be described with reference to method 600 of FIG. 6.

The method 600 may comprise, initially, connecting a sterile liquid transfer port of the fluid device to a corresponding sterile liquid transfer port of another fluid device or cartridge at step 602. The connecting may comprise moving and/or manipulating, by a robot, a fluid device relative to another fluid device or cartridge. For example, alignment features of the sterile liquid transfer ports can be used by the robot to align and couple the sterile liquid transfer ports. In some variations, the robot may move and manipulate the fluid device relative to a sterile liquid transfer instrument of a workcell wherein the other fluid device or cartridge is coupled.

Initially, corresponding ports of the sterile liquid transfer ports can be in a closed configuration, with mechanical seals disposed on surfaces of the corresponding ports providing a primary seal. At step 604, the robot may actuate the corresponding ports of the sterile liquid transfer ports to an open position.

At step 606, a sterilant may be flowed into the sterile liquid transfer port of the fluid device via the fluid conduits. In particular, a sterilant source of the workcell may be coupled to the sterilization process ports of the collar of the fluid device and sterilant may be circulated within the fluid conduits and the sterile liquid transfer port. In some variations, the sterilant may be circulated within the fluid conduits of the fluid device and within the interface formed by the corresponding ports of the connected sterile liquid transfer ports actuated to the open position at step 604. In some variations, the sterilant may be circulated within the fluid conduits and the sterile liquid transfer port for a dwell time of up to about 10 minutes, or between about 1 minute and about 10 minutes, between about 2 minutes and about 9 minutes, between about 3 minutes and about 8 minutes, between about 4 minutes and about 7 minutes, and between about 5 minutes and about 6 minutes, including all ranges and sub-values in-between. In some variations, the sterilant may comprise vaporized hydrogen peroxide having a concentration between about 50% and about 70%, between about 55% and about 65%, between about 56% and about 64%, between about 57% and about 63%, between about 58% and about 62%, and between about 59% and about 61%, including all ranges and sub-values in-between.

Generally, sterilization of a sterile liquid transfer port may comprise one or more steps of dehumidification, conditioning, decontamination, sterilization (with a sterilant), and aeration (e.g., ventilation).

In some variations, step 606 may further comprise dehumidifying the sterile liquid transfer port of the sterile liquid transfer device. For example, pressurized hot air may optionally be circulated within sterile liquid transfer port via the at least one air process port in order to remove residual fluid, moisture, and raise a temperature of inner surfaces of the sterile liquid transfer port.

At step 608, the robot may actuate valves within the sterile liquid transfer ports. Actuation of the valves within the sterile liquid transfer ports transitions a fluid pathway therebetween to an open position. In some variations, the valves may be translated relative to each other. Step 608 may comprise translating a valve of the sterile liquid transfer port of the fluid device relative to a valve of the corresponding sterile liquid transfer port of the other fluid device or cartridge. In some variations, the valves may comprise a spring-loaded shutoff configured to actuate to the open position upon contact with an opposing valve, thereby allowing for fluid communication between the sterile liquid transfer ports. In some variations, each of the valves may comprise corresponding engagement features, such as threading, configured to facilitate coupling between the valves. For example, once the valve of the sterile liquid transfer port of the fluid device is translated into contact with the valve of the corresponding sterile liquid transfer device of the other fluid device or cartridge, the engagement features of the valves may be coupled (e.g., locked) by rotating (e.g., twisting) one of the valves to engage their respective threads to each other. Conversely, one of the valves may be rotated in the opposite direction to uncouple (e.g., unlock) the valves.

After transitioning the valves to the open position to enable fluid transfer between the sterile liquid transfer ports, fluid transfer may begin at step 610. For example, the contents (e.g., fluid, biological material) of the fluid device and the other fluid device or cartridge may be transferred through the sterile liquid transfer ports. The rate and direction of fluid transfer may be determined by fluid conduits, a fluid pump module of the collar of the fluid device, and a corresponding cam of the workcell in communication with a controller of the workcell.

After a desired volume of fluid has been transferred, the fluid conduits and the sterile liquid transfer ports can be purged at step 612. In some variations, compressed air can be provided to the fluid conduits and the sterile liquid transfer ports via the at least one air process port of the collar of the fluid device. By purging the fluid conduits and the sterile liquid transfer ports, method 600 ensures that an entire desired volume of fluid has been transferred.

To begin decoupling the sterile liquid transfer ports at step 614, the valves may be translated away from each other. In some variations, the robot may be configured to manipulate the sterile liquid transfer ports to transition the valves to a closed position and/or to translate the valves away from each other, which may occur simultaneously or independently. The valves in the closed position inhibit fluid flow through the sterile liquid transfer ports. Subsequently, the robot may be configured to transition the ports of the sterile liquid transfer ports to a closed position. Accordingly, the fluid pathway between the sterile liquid transfer ports is closed and the sterile liquid transfer ports may be decoupled at step 616.

Additional detail regarding sterile liquid transfer ports and aspects thereof are provided e.g., in U.S. patent application Ser. No. 17/331,556, entitled “Fluid Connector”, which is incorporated by reference herein.

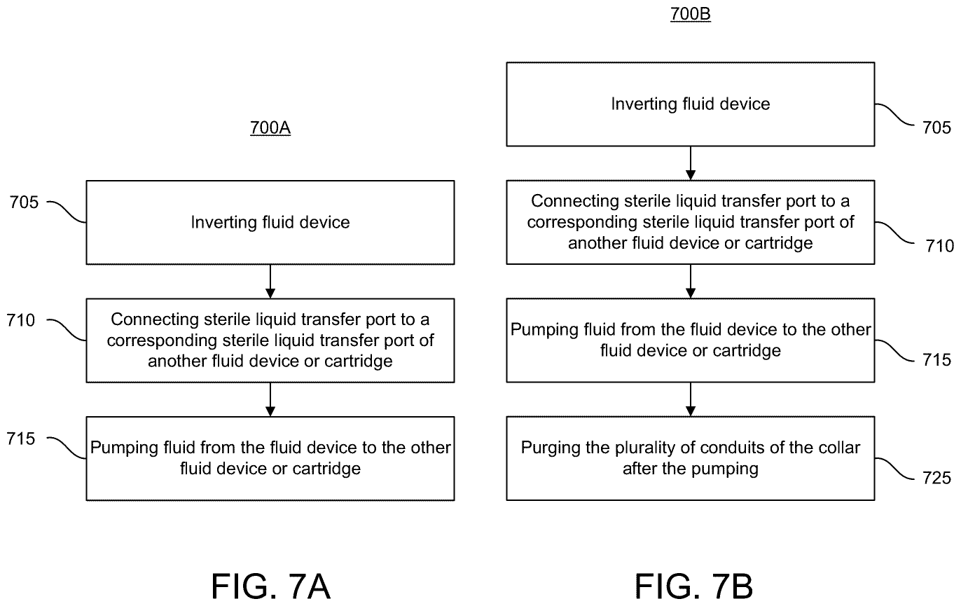

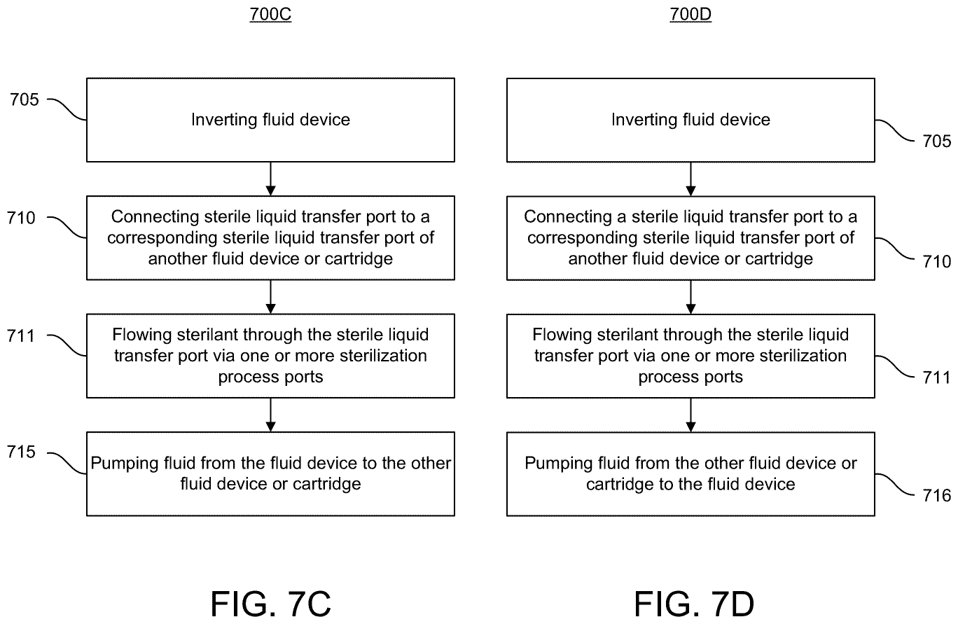

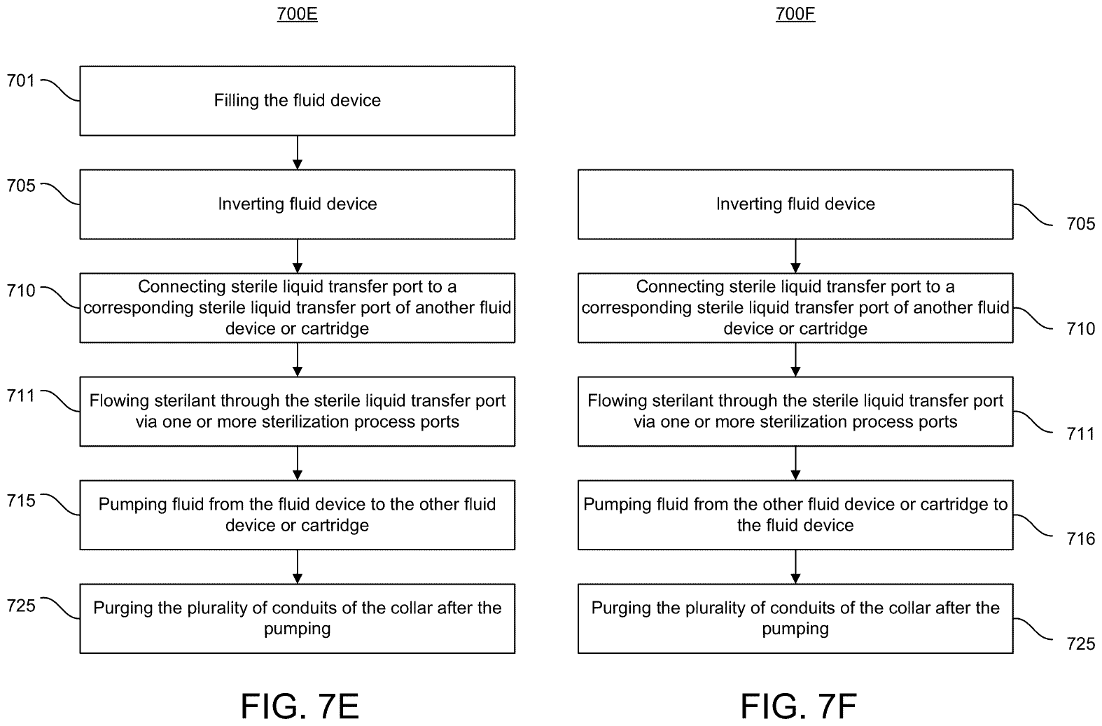

With reference now to FIG. 7A, FIG. 7B, FIG. 7C, FIG. 7D, FIG. 7E, and FIG. 7F, flow diagrams of illustrative variations of methods for automated fluid transfer will be described. It should be appreciated that combinations of the below described methods are also possible without departing from the spirit of the present disclosure. Moreover, it should be appreciated that each of the below described methods may be implemented by a controller of a workcell in concert with, instead of, or independent from a sterile liquid transfer instrument of the workcell.

FIG. 7A is a flow diagram of an illustrative method 700A for automated fluid transfer, where fluid is transferred from a fluid device to another fluid device or cartridge. Such fluid transfer may be necessary to supply e.g., a cartridge with culture medium for a bioreactor module therein.

Initially, the method 700A provides a fluid device pre-filled with fluid. The fluid device may be filled by, e.g., a user within a biosafety cabinet external to the workcell or by a commercial supplier of the fluid. At step 705 of method 700A, the fluid device may be inverted by a robot engaged with the fluid device via one or more engagement features of the fluid device. At step 710, the robot may connect a sterile liquid transfer port of the fluid device with a corresponding sterile liquid transfer port of another fluid device or cartridge. In some variations, this connection can be made within the workcell and/or within a sterile liquid transfer instrument of the workcell. Connecting the sterile liquid transfer ports may include actuation, by the robot, of the ports and valves of the sterile liquid transfer ports, as described above with reference to FIG. 6. At step 715, fluid may be transferred from the fluid device to the other fluid device or cartridge. Specifically, a cam of the workcell may be configured to engage a fluid pump module of the fluid device to control movement of fluid out of the fluid device and into the other fluid device or cartridge via the sterile liquid transfer ports.

FIG. 7B is a flow diagram of an illustrative method 700B for automated fluid transfer, where fluid is transferred from a fluid device to another fluid device or cartridge. Such fluid transfer may be necessary to supply e.g., a cartridge with culture medium for a bioreactor module therein.

Initially, method 700B provides a fluid device pre-filled with fluid. The fluid device may be filled by, e.g., a user within a biosafety cabinet external to the workcell or by a commercial supplier of the fluid. At step 705 of method 700B, the fluid device may be inverted by a robot engaged with the fluid device via one or more engagement features of the fluid device. At step 710, the robot may connect a sterile liquid transfer port of the fluid device with a corresponding sterile liquid transfer port of another fluid device or cartridge. In some variations, this connection can be made within the workcell and/or within a sterile liquid transfer instrument of the workcell. Connecting the sterile liquid transfer ports may include actuation, by the robot, of the ports and valves of the sterile liquid transfer ports, as described above with reference to FIG. 6. At step 715, fluid may be transferred from the fluid device to the other fluid device or cartridge. Specifically, a cam of the workcell may be configured to engage a fluid pump module of the fluid device to control movement of fluid out of the fluid device and into the other fluid device or cartridge via the sterile liquid transfer ports. At step 725, after the fluid transfer via the fluid pump module of the fluid device, compressed air may be provided to the fluid conduits and the sterile liquid transfer port of the fluid device to purge the collar of fluid to be transferred.

FIG. 7C is a flow diagram of an illustrative method 700C for automated fluid transfer, where fluid is transferred from a fluid device to another fluid device or cartridge. Such fluid transfer may be necessary to supply e.g., a cartridge with culture medium for a bioreactor module therein.

Initially, the method 700C provides a fluid device pre-filled with fluid. The fluid device may be filled by, e.g., a user within a biosafety cabinet external to the workcell or by a commercial supplier of the fluid. At step 705 of method 700C, the fluid device may be inverted by a robot engaged with the fluid device via one or more engagement features of the fluid device. At step 710, the robot may connect a sterile liquid transfer port of the fluid device with a corresponding sterile liquid transfer port of another fluid device or cartridge. In some variations, this connection can be made within the workcell and/or within a sterile liquid transfer instrument of the workcell. In some variations, this connection includes actuation of corresponding ports of the connected sterile liquid transfer ports, each of the corresponding ports including a corresponding mechanical seal. At step 711, and prior to actuation of the valves of the connected sterile liquid transfer ports by the robot, sterilant from a sterilant source within the workcell may be circulated within the sterile liquid transfer port and the fluid conduits of the fluid device via sterilization process ports of the fluid device. In some variations, the circulation can include flowing sterilant within the interface formed by the actuated ports of the connected sterile liquid transfer ports. After circulation and removal of the sterilant from the sterile liquid transfer port and the fluid conduits of the fluid device, automated fluid transfer from the fluid device to the other fluid device or cartridge can be performed at step 715 of method 700C. Specifically, a cam of the workcell may be configured to engage a fluid pump module of the fluid device to control movement of fluid out of the fluid device and into the other fluid device or cartridge via the sterile liquid transfer ports.

FIG. 7D is a flow diagram of an illustrative method 700D for automated fluid transfer, where fluid is transferred to a fluid device from another fluid device or cartridge. Such fluid transfer may be necessary to obtain samples of cell solutions for in-process testing, obtain waste fluid for disposal, and the like.

Initially, the method 700D provides an empty fluid device. At step 705 of method 700D, the fluid device may be inverted by a robot engaged with the fluid device via one or more engagement features of the fluid device. At step 710, the robot may connect a sterile liquid transfer port of the fluid device with a corresponding sterile liquid transfer port of another fluid device or cartridge. In some variations, this connection can be made within the workcell and/or within a sterile liquid transfer instrument of the workcell. In some variations, this connection includes actuation of corresponding ports of the connected sterile liquid transfer ports, each of the corresponding ports including a corresponding mechanical seal. At step 711, and prior to actuation of the valves of the connected sterile liquid transfer ports by the robot, sterilant from a sterilant source within the workcell may be circulated within the sterile liquid transfer port and the fluid conduits of the fluid device via sterilization process ports of the fluid device. In some variations, the circulation can include flowing sterilant within the interface formed by the actuated ports of the connected sterile liquid transfer ports. After circulation and removal of the sterilant from the sterile liquid transfer port and the fluid conduits of the fluid device, automated fluid transfer to the fluid device from the other fluid device or cartridge can be performed at step 716 of method 700C. Specifically, a cam of the workcell may be configured to engage a fluid pump module of the fluid device to control movement of fluid into the fluid device from the other fluid device or cartridge via the sterile liquid transfer ports.

In some variations, method 700D may be performed after performance of 700A, 700B, 700C, and/or 700E (to be described below), wherein the fluid transferred to the other fluid device or cartridge from the fluid device and the fluid transferred to the fluid device from the other fluid device or cartridge is associated with the same patient.

FIG. 7E is a flow diagram of an illustrative method 700E for automated fluid transfer, where a fluid device is filled with fluid and then the fluid is transferred from the fluid device to another fluid device or cartridge. Such fluid transfer may be necessary to supply e.g., a cartridge with culture medium for a bioreactor module therein. Moreover, after fluid transfer, the fluid device may be purged to ensure complete fluid transfer.

Initially, the method 700E provides an empty fluid device. At step 701, the fluid device can be filled with a fluid to be transferred to the other fluid device or cartridge. The fluid device can be filled via e.g., a fluid access port of the collar of the fluid device. The filling may occur before or after introduction of the fluid device to the workcell. The amount of fluid filled can be based on a pre-determined volume required by the other fluid device or cartridge. After filling, the fluid device may be inverted by a robot engaged with the fluid device via one or more engagement features of the fluid device at step 705. At step 710, the robot may connect a sterile liquid transfer port of the fluid device with a corresponding sterile liquid transfer port of another fluid device or cartridge. In some variations, this connection can be made within the workcell and/or within a sterile liquid transfer instrument of the workcell. In some variations, this connection includes actuation of corresponding ports of the connected sterile liquid transfer ports, each of the corresponding ports including a corresponding mechanical seal. At step 711, and prior to actuation of the valves of the connected sterile liquid transfer ports by the robot, sterilant from a sterilant source within the workcell may be circulated within the sterile liquid transfer port and the fluid conduits of the fluid device via sterilization process ports of the fluid device. In some variations, the circulation can include flowing sterilant within the interface formed by the actuated ports of the connected sterile liquid transfer ports. After circulation and removal of the sterilant from the sterile liquid transfer port and the fluid conduits of the fluid device, automated fluid transfer from the fluid device to the other fluid device or cartridge can be performed at step 715 of method 700E. Specifically, a cam of the workcell may be configured to engage a fluid pump module of the fluid device to control movement of fluid out of the fluid device and into the other fluid device or cartridge via the sterile liquid transfer ports. At step 725, after the fluid transfer via the fluid pump module of the fluid device, compressed air may be provided to the fluid conduits and the sterile liquid transfer port of the fluid device to purge the collar of fluid to be transferred.

FIG. 7F is a flow diagram of an illustrative method 700F for automated fluid transfer, where a fluid device is initially empty, and fluid is transferred from another fluid device or cartridge to the fluid device. Such fluid transfer may be necessary to obtain samples of cell solutions for in-process testing, obtain waste fluid for disposal, and the like. Moreover, after fluid transfer, the fluid device may be purged to ensure complete fluid transfer.

Initially, the method 700F provides an empty fluid device. At step 705, the fluid device may be inverted by a robot engaged with the fluid device via one or more engagement features of the fluid device. At step 710, the robot may connect a sterile liquid transfer port of the fluid device with a corresponding sterile liquid transfer port of another fluid device or cartridge. In some variations, this connection can be made within the workcell and/or within a sterile liquid transfer instrument of the workcell. In some variations, this connection includes actuation of corresponding ports of the connected sterile liquid transfer ports, each of the corresponding ports including a corresponding mechanical seal. At step 711, and prior to actuation of the valves of the connected sterile liquid transfer ports by the robot, sterilant from a sterilant source within the workcell may be circulated within the sterile liquid transfer port and the fluid conduits of the fluid device via sterilization process ports of the fluid device. In some variations, the circulation can include flowing sterilant within the interface formed by the actuated ports of the connected sterile liquid transfer ports. After circulation and removal of the sterilant from the sterile liquid transfer port and the fluid conduits of the fluid device, automated fluid transfer from the other fluid device or cartridge to the fluid device or cartridge can be performed at step 715 of method 700E. Specifically, a cam of the workcell may be configured to engage a fluid pump module of the fluid device to control movement of fluid into the fluid device from the other fluid device or cartridge via the sterile liquid transfer ports. At step 725, after the fluid transfer via the fluid pump module of the fluid device, compressed air may be provided to the fluid conduits and the sterile liquid transfer port of the fluid device to purge the collar of fluid to be transferred.

In some variations, method 700F may further comprise flowing air into the container of the fluid device, after the fluid transfer, in order to force the transferred fluid out of the fluid device via e.g., the fluid access port of the collar of the fluid device. This subsequent transfer of fluid out of the fluid device can be performed when the fluid device is in any orientation, such as an upright position.

In some variations, the illustrative methods of automated fluid transfer herein further comprise controlling fluid movement within the fluid device using the at least one viewing window of the collar of the fluid device and the sensor(s) and controller of the workcell. In this way, a flow rate of fluid transfer can be controlled so that known volumes of fluid are transferred to and from the fluid device.

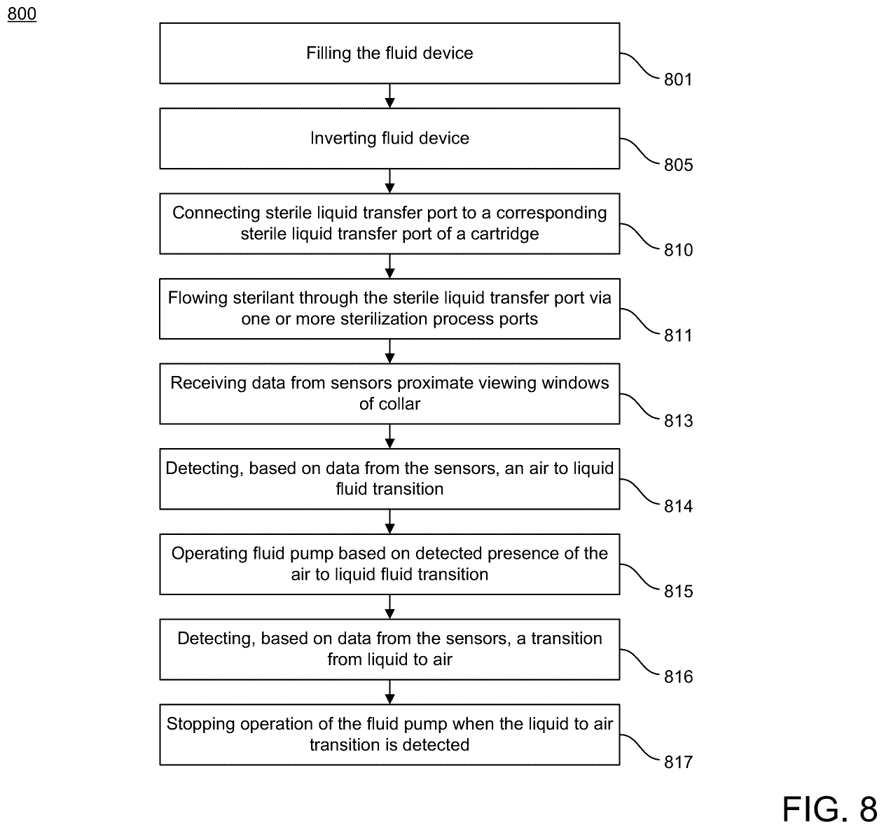

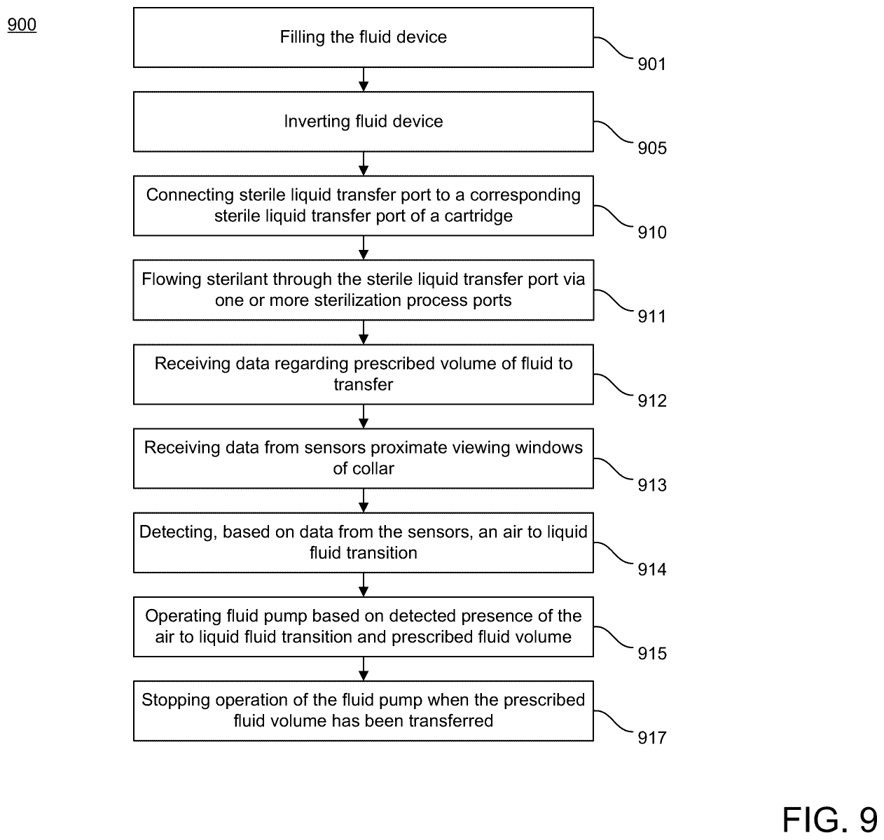

Accordingly, FIG. 8 and FIG. 9 are flow diagrams of other illustrative variations of methods for automated fluid transfer, wherein target transfer volumes and data from the sensor(s) are used to control fluid transfer. It should be appreciated that each of the below described methods may be controlled by a controller of a workcell, in concert with, or independent from, a sterile liquid transfer instrument of the workcell.

With reference first to FIG. 8, method 800 relates to automated fluid transfer at a controlled rate when substantially all of the contents of the fluid device are to be transferred.

Initially, the method 800 provides an empty fluid device. At step 801, the fluid device can be filled with a fluid to be transferred to the other fluid device or cartridge. The filling may occur before or after introduction of the fluid device to the workcell. The amount of fluid filled can be based on a pre-determined volume required by the other fluid device or cartridge. After filling, the fluid device may be inverted by a robot engaged with the fluid device via one or more engagement features of the fluid device at step 805. At step 810, the robot may connect a sterile liquid transfer port of the fluid device with a corresponding sterile liquid transfer port of another fluid device or cartridge. In some variations, this connection can be made within the workcell and/or within a sterile liquid transfer instrument of the workcell. In some variations, this connection includes actuation of corresponding ports of the connected sterile liquid transfer ports, each of the corresponding ports including a corresponding mechanical seal. At step 811, and prior to actuation of the valves of the connected sterile liquid transfer ports by the robot, sterilant from a sterilant source within the workcell may be circulated within the sterile liquid transfer port and the fluid conduits of the fluid device via sterilization process ports of the fluid device. In some variations, the circulation can include flowing sterilant within the interface formed by the actuated ports of the connected sterile liquid transfer ports. After circulation and removal of the sterilant from the sterile liquid transfer port and the fluid conduits of the fluid device, automated fluid transfer from the fluid device to the other fluid device or cartridge can begin. Specifically, a cam of the workcell may be configured to engage a fluid pump module of the fluid device to begin movement of fluid within the fluid device. At step 813, data can be received by the controller from the sensor(s) proximate the at least one viewing window of the collar of the fluid device. As described previously, the at least one viewing window may have a vantage to at least a segment of fluid conduits of the collar of the fluid device. The data received from the sensor(s) may be processed at step 814 to detect when an air to liquid fluid transition occurs within the viewable segment of the fluid conduits. When the transition is detected, and as long as the liquid remains detected by the sensor(s), the fluid pump module and the cam can be operated in a way that transfers the fluid from the fluid device to the other fluid device or cartridge at a controlled flow rate. For example, the cam may be operated at a rate of 60 revolutions per minute. Considered together with a cross sectional area and a length of the fluid conduits and rheological properties of the fluid, the rotational velocity of the cam may correspond to a fluid transfer rate of 100 mL per minute. At step 816, the data received from the sensor(s) may indicate another fluid transition, this time from liquid to air. When the liquid to air transition is detected, it can be assumed that a container of the fluid device is empty. As such, the fluid pump module and cam can be halted at step 817. In some variations, as described above FIG. 7B, FIG. 7E, and FIG. 7F, compressed air may be provided to the fluid conduits and the sterile liquid transfer port of the fluid device after the fluid pumping is stopped at step 817 in order to purge the collar of fluid to be transferred.

With reference now to FIG. 9, method 900 relates to automated fluid transfer at a controlled rate and when a target volume to be transferred is less than a volume of fluid within the fluid device.

Initially, the method 900 provides an empty fluid device. At step 901, the fluid device can be filled with a fluid. The filling may occur before or after introduction of the fluid device to the workcell. The amount of fluid filled can be based on a target volume required by the other fluid device or cartridge. For instance, the volume of the filled fluid may be greater than that which is a target volume to be transferred to the other fluid device or cartridge, thus ensuring that sufficient volume is transferred even if a certain volume of fluid becomes trapped within the fluid device. After filling, the fluid device may be inverted by a robot engaged with the fluid device via one or more engagement features of the fluid device at step 905. At step 910, the robot may connect a sterile liquid transfer port of the fluid device with a corresponding sterile liquid transfer port of another fluid device or cartridge. In some variations, this connection can be made within the workcell and/or within a sterile liquid transfer instrument of the workcell. In some variations, this connection includes actuation of corresponding ports of the connected sterile liquid transfer ports, each of the corresponding ports including a corresponding mechanical seal. At step 911, and prior to actuation of the valves of the connected sterile liquid transfer ports by the robot, sterilant from a sterilant source within the workcell may be circulated within the sterile liquid transfer port and the fluid conduits of the fluid device via sterilization process ports of the fluid device. In some variations, the circulation can include flowing sterilant within the interface formed by the actuated ports of the connected sterile liquid transfer ports. After circulation and removal of the sterilant from the sterile liquid transfer port and the fluid conduits of the fluid device, automated fluid transfer from the fluid device to the other fluid device or cartridge can begin. Specifically, a cam of the workcell may be configured to engage a fluid pump module of the fluid device to begin movement of fluid within the fluid device. Concurrently, or separate from step 911, the target volume, or prescribed volume, to be transferred can be obtained by the controller of the workcell at step 912. After initiating the fluid pump module and the cam to begin movement of fluid within the fluid conduits of the fluid device, data can be received by the controller from the sensor(s) proximate the at least one viewing window of the collar of the fluid device at step 913. As described previously, the at least one viewing window may have a vantage to at least a segment of fluid conduits of the collar of the fluid device. The data received from the sensor(s) may be processed at step 914 to detect when an air to liquid fluid transition occurs within the viewable segment of the fluid conduits. When the transition is detected, the fluid pump module and the cam can be operated in a way that transfers the fluid from the fluid device to the other fluid device or cartridge at a controlled fluid rate and for a controlled duration such that the volume transferred is equal to the target volume received at step 912. For instance, the cam can be operated at a particular rotational velocity and for a particular duration of time based on the target volume, dimensions of the fluid conduits, and rheological properties of the fluid. A temporary halting of the fluid pump module and the cam may also occur prior to performance of step 915 to ensure accuracy and precision in the metered fluid transfer. After the particular duration of time has passed, the fluid pump module and cam can be halted at step 917. In some variations, as described above FIG. 7B, FIG. 7E, and FIG. 7F, compressed air may be provided to the fluid conduits and the sterile liquid transfer port of the fluid device after the fluid pumping is stopped at step 917 in order to purge the collar of fluid to be transferred.

All references cited are herein incorporated by reference in their entirety.

Throughout this application, the term “about” is used to indicate that a value includes the inherent variation of error for the device, or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed herein is:

1. A fluid device for use in automated fluid transfer, comprising:
a container for a volume of fluid; and
a universal collar couplable to the container, the collar comprising
a plurality of conduits,
a sterile liquid transfer port in fluid communication with the plurality of conduits, and
a fluid pump module comprising compressible fluidic tubing coupled between an inlet port and an outlet port, each of the inlet port and the outlet port being in fluid communication with the plurality of conduits, the compressible fluidic tubing being configured to be compressed by a fluid pump to control movement of fluids out of the container.

2. The fluid device of claim 1, wherein the collar further comprises a robot engagement feature.

3. The fluid device of claim 2, wherein the robot engagement feature can be engaged by a robot to manipulate the fluid device.

4. The fluid device of claim 1, wherein the robot engagement feature comprises one or more depressions and/or protrusions within a surface of the collar.

5. The fluid device of claim 1, wherein the collar is releasably couplable to the container via corresponding features disposed on the collar and on the container.

6. The fluid device of claim 1, wherein a fluid capacity of the container ranges between about 1 milliliter to about 1 liter.

7. The fluid device of claim 1, wherein the control of the movement of the fluids comprises bidirectional movement control.

8. The fluid device of claim 1, wherein the collar further comprises one or more sterilization process ports in fluid communication with the plurality of conduits.

9. The fluid device of claim 8, wherein sterilant provided via the one or more sterilization process ports comprises one or more of vaporized hydrogen peroxide, ionized hydrogen peroxide, chlorine dioxide, and ethylene oxide.

10. The fluid device of claim 8, wherein the one or more sterilization process ports are valved.

11. The fluid device of claim 8, wherein the one or more sterilization process ports are coupled to one or more passive valves.

12. The fluid device of claim 1, wherein the collar further comprises one or more air process ports in fluid communication with the plurality of conduits.

13. The fluid device of claim 12, wherein the one or more air process ports are compressed air process ports.

14. The fluid device of claim 1, wherein the collar further comprises valves to prevent the introduction of air into fluids during the fluid transfer.

15. The fluid device of claim 12, wherein the one or more air process ports are air vents.

16. The fluid device of claim 12, wherein the collar further comprises hydrophobic filters couplable to the one or more air process ports.

17. The fluid device of claim 1, wherein the collar further comprises a fluid access port for filling the container after coupling of the collar and the container.

18. The fluid device of claim 1, wherein the collar further comprises viewing windows that permit optical evaluation of fluid within a fluid conduit of the collar.

19. The fluid device of claim 1, wherein the sterile liquid transfer port further comprises a mechanical seal.

20. The fluid device of claim 19, wherein the collar further comprises one or more sterilization process ports in fluid communication with the plurality of conduits, and wherein the mechanical seal of the sterile liquid transfer port and a sterilant provided via the one or more sterilization process ports ensure sterility of the collar.

21. The fluid device of claim 1, wherein the collar further comprises a pressure relief valve at the outlet port of the compressible fluidic tubing of the fluid pump module, and wherein the outlet port is further in fluid communication with the container such that, when there is excessive pressure at the outlet port, liquid is flowed into the container.

22. The fluid device of claim 1, wherein the container comprises an opening, and wherein the collar further comprises a fluid transport feature couplable to the opening and in fluid communication with the plurality of conduits.

23. The fluid device of claim 22, wherein the fluid transport feature of the collar comprises a venting tube configured to extend through the opening of the container and to be disposed within the container.

24. The fluid device of claim 23, wherein the venting tube further comprises a liquid vent reservoir configured to capture, upon inversion of the fluid device, fluid trapped within the venting tube.

25. The fluid device of claim 24, wherein a volume of the liquid vent reservoir is larger than a volume of the venting tube within the container.

26. The fluid device of claim 23, wherein the fluid transport feature further comprises a liquid flow tube configured to extend through the opening of the container and to be disposed within the container.

27. The fluid device of claim 26, wherein the collar further comprises one or more air process ports and the one or more air process ports are in fluid communication with the liquid flow tube.

28. The fluid device of claim 23, wherein the fluid transport feature further comprises a flow port in fluid communication with the container and the sterile liquid transfer port.

29. The fluid device of claim 1, wherein the sterile liquid transfer port further comprises a mechanical seal and the collar further comprises one or more sterilization ports in fluid communication with the plurality of conduits, the mechanical seal providing a first mechanism to achieve sterile sterilization and sterilant provided via the one or more sterilization ports providing a second mechanism to achieve sterilization.

30. A method for automated fluid transfer, the method comprising:
inverting, by a robot, a fluid device comprising a container and a universal collar comprising a plurality of conduits and a sterile liquid transfer port in fluid communication with the plurality of conduits;
connecting, by the robot, the sterile liquid transfer port of the inverted fluid device to a corresponding sterile liquid transfer port of a cartridge; and
pumping, via the plurality of conduits and the sterile liquid transfer port, fluid from the fluid device to the cartridge.

31. The method of claim 30, further comprising sterilizing, after the connecting and before the pumping, the sterile liquid transfer port via one or more sterilization process ports of the collar in fluid communication with the plurality of conduits.

32. The method of claim 30, further comprising
actuating, by the robot and after the sterilizing, a valve of each of the sterile liquid transfer port and the corresponding sterile liquid transfer port to permit the pumping.

33. The method of claim 30, wherein the inverting comprises
engaging, by the robot, a robot engagement feature of the collar of the fluid device.

34. A method for automated fluid transfer, the method comprising:
inverting, by a robot, a fluid device comprising a container and a universal collar comprising a plurality of conduits, a sterile liquid transfer port, and an air process port, each of the sterile liquid transfer port and the air process port being in fluid communication with the plurality of conduits;
connecting, by the robot, the sterile liquid transfer port of the inverted fluid device to a corresponding sterile liquid transfer port of a cartridge;
pumping, via the plurality of conduits and the sterile liquid transfer port, at least a portion of a fluid from the inverted fluid device to the cartridge; and
purging the plurality of conduits after the pumping using compressed air via the air process port.

35. A method for automated fluid transfer, the method comprising:
inverting, by a robot, a fluid device comprising a container and a universal collar comprising a robot engagement feature, a plurality of conduits, a sterile liquid transfer port, and a plurality of sterilization process ports, each of the sterile liquid transfer port and the plurality of sterilization process ports being in fluid communication with the plurality of conduits;
connecting, by the robot, the sterile liquid transfer port of the inverted fluid device to a corresponding sterile liquid transfer port of a cartridge;
flowing sterilant through the sterile liquid transfer port via the one or more sterilization process ports; and
pumping, via the plurality of conduits and the sterile liquid transfer port, at least a portion of a fluid from the inverted fluid device to the cartridge.

36. A method for automated fluid transfer, the method comprising:
filling, when the fluid device is in an upright position, a fluid device comprising a container and a universal collar comprising a robot engagement feature, a plurality of conduits, and a sterile liquid transfer port;
inverting, by a robot via the robot engagement feature, the fluid device;
connecting, by the robot, the sterile liquid transfer port of the inverted fluid device to a corresponding sterile liquid transfer port of a cartridge; and
pumping, via the plurality of conduits and the sterile liquid transfer device, at least a portion of a fluid from the fluid device to the cartridge.

37. The method of claim 36, further comprising
purging, via an air process port of the collar, the plurality of conduits within the collar.

38. The method of claim 36, wherein the filling is performed via a fluid access port of the collar.

39. The method of claim 36, wherein the pumping further comprises
receiving data from sensors arranged proximate to viewing windows of the collar, the sensors configured to detect the presence of liquid within a segment of the plurality of conduits;
detecting, based on the received data, an air to liquid fluid transition;
operate fluid pump based on the detected presence of the air to liquid fluid transition;
detecting, based on the received data, a liquid to air fluid transition; and
stopping operation of the fluid pump when the liquid to air fluid transition is detected.

40. The method of claim 36, wherein the pumping further comprises receiving data regarding a prescribed volume of fluid to transfer to the cartridge;
receiving data from sensors arranged proximate to viewing windows of the collar, the sensors configured to detect the presence of liquid within a segment of the plurality of conduits;
detecting, based on the received data, an air to liquid fluid transition;
operate fluid pump based on the detected presence of the air to liquid fluid transition and the received data regarding the prescribed volume of fluid; and
stopping operation of the fluid pump when the prescribed volume of fluid has been transferred.

41. A system for automated fluid transfer, comprising:
a fluid pump;
a fluid device, comprising
a container for a volume of fluid, and
a universal collar couplable to the container, the collar comprising
a plurality of conduits,
a sterile liquid transfer port in fluid communication with the plurality of conduits,
a fluid pump module comprising compressible fluidic tubing coupled between an inlet port and an outlet port, each of the inlet port and the outlet port being in fluid communication with the plurality of conduits, the compressible fluidic tubing being configured to be compressed by the fluid pump to control movement of fluids out of the container, and
one or more viewing windows; and
one or more sensors configured to detect, via the one or more viewing windows, the presence of liquid within a segment of the plurality of conduits.

42. The system of claim 41, further comprising:
a processor configured to
receive data from the one or more sensors;
detect, based on the received data, a fluid transition from air to liquid;
start the fluid pump;
detect a fluid transition from liquid to air; and
stop the fluid pump when the fluid transition from liquid to air is detected.

43. A system for automated fluid transfer, comprising:
a fluid pump;
a robot; and
a fluid device, comprising
a container for a volume of fluid, and
a universal collar couplable to the container, the collar comprising
a robot engagement feature engageable by the robot,
a plurality of conduits,
a sterile liquid transfer port in fluid communication with the plurality of conduits,
a fluid pump module comprising compressible fluidic tubing coupled between an inlet port and an outlet port, each of the inlet port and the outlet port being in fluid communication with the plurality of conduits, the compressible fluidic tubing being configured to be compressed by the fluid pump to control movement of fluids out of the container.

44. A method for automated fluid transfer, the method comprising:
connecting, by a robot, a sterile liquid transfer port of a fluid device to a corresponding sterile liquid transfer port of a cartridge;
pumping, via a plurality of conduits of the fluid device and the sterile liquid transfer port, at least a portion of a fluid from the fluid device to the cartridge; and
purging the plurality of conduits after the pumping using compressed air via an air process port of the fluid device.

45. A fluid device for automated fluid transfer, comprising:
a container for a volume of fluid; and
a universal collar couplable to the container, the collar comprising:
a plurality of conduits;
a sterile liquid transfer port in fluid communication with the plurality of conduits;
a fluid pump module comprising compressible fluidic tubing coupled between an inlet port and an outlet port, each of the inlet port and the outlet port being in fluid communication with the plurality of conduits, the compressible fluidic tubing being configured to be compressed by the fluid pump to control movement of fluids out of the container;
an air process port; and
a ball valve coupled to the air process port, wherein the fluid device is configured to be positioned in an upright orientation and an inverted orientation, and wherein the ball valve is configured to prevent the fluid of the container from flowing within the air process port when the fluid device is in the inverted orientation.
